SEQUENCE LISTING (9 sequences)

SEQ ID NO: 1 - protein, 472 aa, Homo sapiens
MGSKMNLIEHSHLPTTDEFSFSENLFGVLTEQVAGPLGQNLEVEPYSQYSNVQFPQVQPQISSSSYYSNLGFYPQQPEEWYSPGIYELRRMPAETLYQGETEVAEMPVTKKPRMGASAGRIKGDELCVVCGDRASGYHYNALTCEGCKGFFRRSITKNAVYKCKNGGNCVMDMYMRRKCQECRLRKCKEMGMLAECLLTEIQCKSKRLRKNVKQHADQTVNEDSEGRDLRQVTSTTKSCREKTELTPDQQTLLHFIMDSYNKQRMPQEITNKILKEEFSAEENFLILTEMATNHVQVLVEFTKKLPGFQTLDHEDQIALLKGSAVEAMFLRSAEIFNKKLPSGHSDLLEERIRNSGISDEYITPMFSFYKSIGELKMTQEEYALLTAIVILSPDRQYIKDREAVEKLQEPLLDVLQKLCKIHQPENPQHFACLLGRLTELRTFNHHHAEMLMSWRVNDHKFTPLLCEIWDVQ

SEQ ID NO: 2 - protein, 472 aa, Homo sapiens; identical to SEQ ID NO: 1

SEQ ID NO: 3 - protein, 476 aa, Homo sapiens
MGSKMNLIEHSHLPTTDEFSFSENLFGVLTEQVAGPLGQNLEVEPYSQYSNVQFPQVQPQISSSSYYSNLGFYPQQPEEWYSPGIYELRRMPAETLYQGETEVAEMPVTKKPRMGASAGRIKGDELCVVCGDRASGYHYNALTCEGCKGFFRRSITKNAVYKCKNGGNCVMDMYMRRKCQECRLRKCKEMGMLAECMYTGLLTEIQCKSKRLRKNVKQHADQTVNEDSEGRDLRQVTSTTKSCREKTELTPDQQTLLHFIMDSYNKQRMPQEITNKILKEEFSAEENFLILTEMATNHVQVLVEFTKKLPGFQTLDHEDQIALLKGSAVEAMFLRSAEIFNKKLPSGHSDLLEERIRNSGISDEYITPMFSFYKSIGELKMTQEEYALLTAIVILSPDRQYIKDREAVEKLQEPLLDVLQKLCKIHQPENPQHFACLLGRLTELRTFNHHHAEMLMSWRVNDHKFTPLLCEIWDVQ

SEQ ID NO: 4 - protein, 482 aa, Homo sapiens
MVMQFQGLENPIQISPHCSCTPSGFFMEMMSMKPAKGVLTEQVAGPLGQNLEVEPYSQYSNVQFPQVQPQISSSSYYSNLGFYPQQPEEWYSPGIYELRRMPAETLYQGETEVAEMPVTKKPRMGASAGRIKGDELCVVCGDRASGYHYNALTCEGCKGFFRRSITKNAVYKCKNGGNCVMDMYMRRKCQECRLRKCKEMGMLAECLLTEIQCKSKRLRKNVKQHADQTVNEDSEGRDLRQVTSTTKSCREKTELTPDQQTLLHFIMDSYNKQRMPQEITNKILKEEFSAEENFLILTEMATNHVQVLVEFTKKLPGFQTLDHEDQIALLKGSAVEAMFLRSAEIFNKKLPSGHSDLLEERIRNSGISDEYITPMFSFYKSIGELKMTQEEYALLTAIVILSPDRQYIKDREAVEKLQEPLLDVLQKLCKIHQPENPQHFACLLGRLTELRTFNHHHAEMLMSWRVNDHKFTPLLCEIWDVQ

SEQ ID NO: 5 - protein, 486 aa, Homo sapiens
MVMQFQGLENPIQISPHCSCTPSGFFMEMMSMKPAKGVLTEQVAGPLGQNLEVEPYSQYSNVQFPQVQPQISSSSYYSNLGFYPQQPEEWYSPGIYELRRMPAETLYQGETEVAEMPVTKKPRMGASAGRIKGDELCVVCGDRASGYHYNALTCEGCKGFFRRSITKNAVYKCKNGGNCVMDMYMRRKCQECRLRKCKEMGMLAECMYTGLLTEIQCKSKRLRKNVKQHADQTVNEDSEGRDLRQVTSTTKSCREKTELTPDQQTLLHFIMDSYNKQRMPQEITNKILKEEFSAEENFLILTEMATNHVQVLVEFTKKLPGFQTLDHEDQIALLKGSAVEAMFLRSAEIFNKKLPSGHSDLLEERIRNSGISDEYITPMFSFYKSIGELKMTQEEYALLTAIVILSPDRQYIKDREAVEKLQEPLLDVLQKLCKIHQPENPQHFACLLGRLTELRTFNHHHAEMLMSWRVNDHKFTPLLCEIWDVQ

SEQ ID NO: 6 - DNA, 2218 bp, Homo sapiens
acgagactctctcctcctcctcacctcattgtctccccgacttatcctaatgcgaaattggattctgagcatttgtagcaaaatcgctgggatctggagaggaagactcagtccagaatcctcccagggccttgaaagtccatctctgacccaaaacaatccaaggaggtagaagacatcgtagaaggagtgaaagaagaaaagaagacttagaaacatagctcaaagtgaacactgcttctcttagtttcctggatttcttctggacatttcctcaagatgaaacttcagacactttggagttttttttgaagaccaccataaagaaagtgcatttcaattgaaaaatttggatgggatcaaaaatgaatctcattgaacattcccatttacctaccacagatgaattttctttttctgaaaatttatttggtgttttaacagaacaagtggcaggtcctctgggacagaacctggaagtggaaccatactcgcaatacagcaatgttcagtttccccaagttcaaccacagatttcctcgtcatcctattattccaacctgggtttctacccccagcagcctgaagagtggtactctcctggaatatatgaactcaggcgtatgccagctgagactctctaccagggagaaactgaggtagcagagatgcctgtaacaaagaagccccgcatgggcgcgtcagcagggaggatcaaaggggatgagctgtgtgttgtttgtggagacagagcctctggataccactataatgcactgacctgtgaggggtgtaaaggtttcttcaggagaagcattaccaaaaacgctgtgtacaagtgtaaaaacgggggcaactgtgtgatggatatgtacatgcgaagaaagtgtcaagagtgtcgactaaggaaatgcaaagagatgggaatgttggctgaatgcttgttaactgaaattcagtgtaaatctaagcgactgagaaaaaatgtgaagcagcatgcagatcagaccgtgaatgaagacagtgaaggtcgtgacttgcgacaagtgacctcgacaacaaagtcatgcagggagaaaactgaactcaccccagatcaacagactcttctacattttattatggattcatataacaaacagaggatgcctcaggaaataacaaataaaattttaaaagaagaattcagtgcagaagaaaattttctcattttgacggaaatggcaaccaatcatgtacaggttcttgtagaattcacaaaaaagctaccaggatttcagactttggaccatgaagaccagattgctttgctgaaagggtctgcggttgaagctatgttccttcgttcagctgagattttcaataagaaacttccgtctgggcattctgacctattggaagaaagaattcgaaatagtggtatctctgatgaatatataacacctatgtttagtttttataaaagtattggggaactgaaaatgactcaagaggagtatgctctgcttacagcaattgttatcctgtctccagatagacaatacataaaggatagagaggcagtagagaagcttcaggagccacttcttgatgtgctacaaaagttgtgtaagattcaccagcctgaaaatcctcaacactttgcctgtctcctgggtcgcctgactgaattacggacattcaatcatcaccacgctgagatgctgatgtcatggagagtaaacgaccacaagtttaccccacttctctgtgaaatctgggacgtgcagtgatggggattacaggggaggggtctagctcctttttctctctcatattaatctgatgtataactttcctttatttcacttgtacccagtttcactcaagaaatcttgatgaatatttatgttgtaattacatgtgtaacttccacaactgtaaatattgggctagatagaacaactttctctacattgtgttttaaaaggctccagggaatcctgcattctaattggcaagccctgtttgcctaattaaattgattgttacttcaattctatctgttgaactagggaaaatctcattttgctcatcttaccatattgcatatattttattaaagagttgtattcaatcttggcaataaagcaaacataatggcaacagaaaaaaaaaaaaaaaaaaaaaaaaaaaaaaaaaaaaaaaaaaaaaaaaaaaaaaaaaaaaaaaaaaaaaa

SEQ ID NO: 7 - DNA, 1431 bp, Homo sapiens
atgggatcaaaaatgaatctcattgaacattcccatttacctaccacagatgaattttctttttctgaaaatttatttggtgttttaacagaacaagtggcaggtcctctgggacagaacctggaagtggaaccatactcgcaatacagcaatgttcagtttccccaagttcaaccacagatttcctcgtcatcctattattccaacctgggtttctacccccagcagcctgaagagtggtactctcctggaatatatgaactcaggcgtatgccagctgagactctctaccagggagaaactgaggtagcagagatgcctgtaacaaagaagccccgcatgggcgcgtcagcagggaggatcaaaggggatgagctgtgtgttgtttgtggagacagagcctctggataccactataatgcactgacctgtgaggggtgtaaaggtttcttcaggagaagcattaccaaaaacgctgtgtacaagtgtaaaaacgggggcaactgtgtgatggatatgtacatgcgaagaaagtgtcaagagtgtcgactaaggaaatgcaaagagatgggaatgttggctgaatgtatgtatacaggcttgttaactgaaattcagtgtaaatctaagcgactgagaaaaaatgtgaagcagcatgcagatcagaccgtgaatgaagacagtgaaggtcgtgacttgcgacaagtgacctcgacaacaaagtcatgcagggagaaaactgaactcaccccagatcaacagactcttctacattttattatggattcatataacaaacagaggatgcctcaggaaataacaaataaaattttaaaagaagaattcagtgcagaagaaaattttctcattttgacggaaatggcaaccaatcatgtacaggttcttgtagaattcacaaaaaagctaccaggatttcagactttggaccatgaagaccagattgctttgctgaaagggtctgcggttgaagctatgttccttcgttcagctgagattttcaataagaaacttccgtctgggcattctgacctattggaagaaagaattcgaaatagtggtatctctgatgaatatataacacctatgtttagtttttataaaagtattggggaactgaaaatgactcaagaggagtatgctctgcttacagcaattgttatcctgtctccagatagacaatacataaaggatagagaggcagtagagaagcttcaggagccacttcttgatgtgctacaaaagttgtgtaagattcaccagcctgaaaatcctcaacactttgcctgtctcctgggtcgcctgactgaattacggacattcaatcatcaccacgctgagatgctgatgtcatggagagtaaacgaccacaagtttaccccacttctctgtgaaatctgggacgtgcagtga

SEQ ID NO: 8 - DNA, 1477 bp, Homo sapiens
attttcagtggctgtgaataagctaagaatggtaatgcagtttcaggggttagaaaatccaattcaaattagtcctcactgcagctgtacgccgtcaggatttttcatggaaatgatgagtatgaagcccgcgaaaggtgttttaacagaacaagtggcaggtcctctgggacagaacctggaagtggaaccatactcgcaatacagcaatgttcagtttccccaagttcaaccacagatttcctcgtcatcctattattccaacctgggtttctacccccagcagcctgaagagtggtactctcctggaatatatgaactcaggcgtatgccagctgagactctctaccagggagaaactgaggtagcagagatgcctgtaacaaagaagccccgcatgggcgcgtcagcagggaggatcaaaggggatgagctgtgtgttgtttgtggagacagagcctctggataccactataatgcactgacctgtgaggggtgtaaaggtttcttcaggagaagcattaccaaaaacgctgtgtacaagtgtaaaaacgggggcaactgtgtgatggatatgtacatgcgaagaaagtgtcaagagtgtcgactaaggaaatgcaaagagatgggaatgttggctgaatgcttgttaactgaaattcagtgtaaatctaagcgactgagaaaaaatgtgaagcagcatgcagatcagaccgtgaatgaagacagtgaaggtcgtgacttgcgacaagtgacctcgacaacaaagtcatgcagggagaaaactgaactcaccccagatcaacagactcttctacattttattatggattcatataacaaacagaggatgcctcaggaaataacaaataaaattttaaaagaagaattcagtgcagaagaaaattttctcattttgacggaaatggcaaccaatcatgtacaggttcttgtagaattcacaaaaaagctaccaggatttcagactttggaccatgaagaccagattgctttgctgaaagggtctgcggttgaagctatgttccttcgttcagctgagattttcaataagaaacttccgtctgggcattctgacctattggaagaaagaattcgaaatagtggtatctctgatgaatatataacacctatgtttagtttttataaaagtattggggaactgaaaatgactcaagaggagtatgctctgcttacagcaattgttatcctgtctccagatagacaatacataaaggatagagaggcagtagagaagcttcaggagccacttcttgatgtgctacaaaagttgtgtaagattcaccagcctgaaaatcctcaacactttgcctgtctcctgggtcgcctgactgaattacggacattcaatcatcaccacgctgagatgctgatgtcatggagagtaaacgaccacaagtttaccccacttctctgtgaaatctgggacgtgcagtga

SEQ ID NO: 9 - DNA, 1489 bp, Homo sapiens
attttcagtggctgtgaataagctaagaatggtaatgcagtttcaggggttagaaaatccaattcaaattagtcctcactgcagctgtacgccgtcaggatttttcatggaaatgatgagtatgaagcccgcgaaaggtgttttaacagaacaagtggcaggtcctctgggacagaacctggaagtggaaccatactcgcaatacagcaatgttcagtttccccaagttcaaccacagatttcctcgtcatcctattattccaacctgggtttctacccccagcagcctgaagagtggtactctcctggaatatatgaactcaggcgtatgccagctgagactctctaccagggagaaactgaggtagcagagatgcctgtaacaaagaagccccgcatgggcgcgtcagcagggaggatcaaaggggatgagctgtgtgttgtttgtggagacagagcctctggataccactataatgcactgacctgtgaggggtgtaaaggtttcttcaggagaagcattaccaaaaacgctgtgtacaagtgtaaaaacgggggcaactgtgtgatggatatgtacatgcgaagaaagtgtcaagagtgtcgactaaggaaatgcaaagagatgggaatgttggctgaatgtatgtatacaggcttgttaactgaaattcagtgtaaatctaagcgactgagaaaaaatgtgaagcagcatgcagatcagaccgtgaatgaagacagtgaaggtcgtgacttgcgacaagtgacctcgacaacaaagtcatgcagggagaaaactgaactcaccccagatcaacagactcttctacattttattatggattcatataacaaacagaggatgcctcaggaaataacaaataaaattttaaaagaagaattcagtgcagaagaaaattttctcattttgacggaaatggcaaccaatcatgtacaggttcttgtagaattcacaaaaaagctaccaggatttcagactttggaccatgaagaccagattgctttgctgaaagggtctgcggttgaagctatgttccttcgttcagctgagattttcaataagaaacttccgtctgggcattctgacctattggaagaaagaattcgaaatagtggtatctctgatgaatatataacacctatgtttagtttttataaaagtattggggaactgaaaatgactcaagaggagtatgctctgcttacagcaattgttatcctgtctccagatagacaatacataaaggatagagaggcagtagagaagcttcaggagccacttcttgatgtgctacaaaagttgtgtaagattcaccagcctgaaaatcctcaacactttgcctgtctcctgggtcgcctgactgaattacggacattcaatcatcaccacgctgagatgctgatgtcatggagagtaaacgaccacaagtttaccccacttctctgtgaaatctgggacgtgcagtga

(19) United States
(12) Patent Application Publication
(10) Pub. No.: US 2010/0144690 A1
(43) Pub. Date: Jun. 10, 2010

(54) MEDICAMENTS AND METHODS FOR PROMOTING WOUND CONTRACTION

(75) Inventors: Mark W. J. Ferguson, Manchester (GB); Nicholas Occleston, Manchester (GB); Sharon O'Kane, Manchester (GB); Nicholas Goldspink, Manchester (GB); Kerry Nield, Manchester (GB)

Correspondence Address: FOLEY AND LARDNER LLP;SUITE 500, 3000 K STREET NW, WASHINGTON, DC, 20007, US

(73) Assignee: RENOVO LIMITED

(21) Appl. No.: 12/520,496

(22) PCT Filed: Dec. 23, 2007
(86) PCT No.: PCT/GB07/04951

§ 371 Date: Jun. 19, 2009

(30) Foreign Application Priority Data
Dec. 23, 2006 (GB) 0625966.7

(51) Int. Cl. A61K 31/57; A61K 31/575; A61P 17/02

(52) U.S. Cl. 514/177; 514/182

(57) ABSTRACT

Provided are antagonists of FXR activity for use as medicament for the prevention, reduction or inhibition of scarring. This use may preferably be to prevent, reduce or inhibit scarring formed on healing of wounds. The invention also provides corresponding methods of treatment. Preferred antagonists of FXR activity include guggulsterone (Z); guggulsterone (E); a scalarane; 80-574; and a 5α-bile alcohol. In advantageous embodiments, up to 32 μM of the antagonist of FXR activity may be provided per linear cm of wound, or cm² of a wound or fibrotic disorder, over a 24 hour period in order to inhibit scarring.

The present invention relates to medicaments and methods for the prevention, reduction or inhibition of scarring. The invention also provides medicaments and methods for the promotion of wound contraction.

A scar may be defined as “fibrous connective tissue that forms at the site of injury or disease in any tissue of the body” (the scarring response is common throughout all adult mammals). Scarring may result from healing of a wound, or through the deposition of scar tissue associated with fibrotic disorders. The scarring response is conserved between the majority of tissue types and in each case leads to the same result, formation of fibrotic tissue termed “a scar”. Many different processes are at work during the scarring response, and much research has been conducted into discovering what mediates these processes, and how they interact with each other to produce the final outcome.

The scarring response has arisen as the evolutionary solution to the biological imperative to prevent the death of a wounded animal. Thus, to overcome the risk of mortality due to infection or blood loss, the body reacts rapidly to repair the damaged area, rather than attempt to regenerate the damaged tissue.

In the case of a scar that results from healing of a wound, the scar may be defined as the structure produced as a result of the reparative response. Since the injured tissue is not regenerated to attain the same tissue architecture present before wounding, a scar may be identified by virtue of its abnormal morphology as compared to unwounded tissue. Such scars are composed of connective tissue deposited during the healing process. A scar may comprise connective tissue that has an abnormal organisation (as seen in scars of the skin) and/or connective tissue that is present in an abnormally increased amount. Most scars consist of both abnormally organised and excess connective tissue.

The abnormal structure of scars may be observed with reference to both their internal structure (which may be determined by means of microscopic analysis) and their external appearance (which may be assessed macroscopically). Extracellular matrix (ECM) molecules comprise the major structural component of both “normal” (unwounded) and scarred skin. In normal skin these molecules form fibres that have a characteristic random arrangement that is commonly referred to as “basket-weave”. In general the fibres observed within normal skin are of larger diameter than those seen in scars. Fibres in scars also exhibit a marked degree of alignment with each other as compared to the random arrangement of fibres in normal skin. Both the size and arrangement of ECM may contribute to the scars altered mechanical properties, most notably increased stiffness, when compared with normal skin.

Viewed macroscopically, scars may be depressed below the surface of the surrounding tissue, or elevated above the surface of the undamaged skin. Scars may be relatively darker coloured than the normal skin (hyperpigmentation) or may have a paler colour (hypopigmentation) than their surroundings. Either hyperpigmented or hypopigmented scars constitute a readily apparent cosmetic defect. It is also known that scars may be redder than unwounded skin, causing them to be noticeable and cosmetically unacceptable. It has been shown that the cosmetic appearance of a scar is one of the major factors contributing to the psychological impact of scars upon the sufferer, and that these effects can remain long after the cause of the scar, be it either a wound or a fibrotic disorder, has passed.

Scars may also have deleterious physical effects upon the sufferer. These effects typically arise as a result of the mechanical differences between scars and normal skin. The abnormal structure and composition of scars mean that they are typically less flexible than normal skin. As a result scars may be responsible for impairment of normal function (such as in the case of scars covering joints which may restrict the possible range of movement) and may retard normal growth if present from an early age.

Scarring may also occur at many other body sites, in addition to the skin, and the effects of scarring at these sites may also be deleterious to the sufferer. For example, scarring in the eye (whether as a result of accidental injury, surgical intervention, or a fibrotic disorder) can impair vision and even lead to blindness. Scarring of the internal organs may lead to the formation of strictures and adhesions that significantly or totally impair function of the organ in question. Scarring of tendons and ligaments may cause lasting damage to these organs, and thereby reduce the motility or function of associated joints. Scarring associated with blood vessels, and particularly the valves of the heart, may occur after injury or surgery. Scarring of blood vessels may lead to restenosis, which causes a narrowing of the blood vessel and thus reduces the flow of blood through the scarred area. Scarring in the central or peripheral nervous system may prevent transmission along the nerve and may prevent or reduce reconnection of damaged nerve tissue.

The effects outlined above may all arise as a result of the normal progression of the wound healing response (in the case of scars that result from healing of a wound). There are, however, many ways in which the scarring response may be abnormally altered; and these are frequently associated with even more damaging results.

One way in which the scarring response may be altered is through the production of abnormal excessive scarring (commonly referred to as pathological scarring).

Hypertrophic scars are a common form of pathological scarring, and have marked adverse effects on the sufferer. Hypertrophic scars are elevated above the normal surface of the skin and contain excessive collagen arranged in an abnormal pattern. As a result, such scars are often associated with a marked loss of normal mechanical function. This may be exacerbated by the tendency of hypertrophic scars to undergo contraction after their formation, an activity normally ascribed to their abnormal expression of muscle-related proteins (particularly smooth-muscle actin). Children suffer from an increased likelihood of hypertrophic scar formation, particularly as a result of burn injuries.

Keloids are another common form of pathological scarring. Keloid scars are not only elevated above the surface of the skin but also extend beyond the boundaries of the original injury. Keloids contain excessive connective tissue that is organised in an abnormal fashion, normally manifested as whorls of collagenous tissue. The causes of keloid formation are open to conjecture, but it is generally recognised that some individuals have a genetic predisposition to their formation. Both hypertrophic scars and keloids are particularly common in those of the African Continental Ancestry Group or Asian Continental Ancestry Group.

A further common form of pathological scarring is pterygium in which a wedge-shaped fibrotic outgrowth of subconjunctival tissue may grow to the border of the cornea or beyond. Pterygium is more frequent among those frequently exposed to strong sunlight or dusty conditions.

Although scarring may be defined as the production of the structure that remains following the healing of a wound, similar disturbances of the extracellular matrix may also give rise to scarring associated with a number of medical conditions known as fibrotic disorders. In these disorders excessive fibrosis leads to pathological derangement and malfunctioning of tissue. Scars associated with fibrotic disorders are characterised by the accumulation of fibrous tissue (predominately collagens, as described above) in an abnormal fashion within the damaged tissue. Accumulation of such fibrous tissues may result from a variety of disease processes, all of which lead to the same end result. The biological and pathological processes underlying the development of scars associated with fibrotic disorders are generally sufficiently similar to those involved in the formation of scars resulting from healing of a wound that those compounds that may be used to prevent, reduce or inhibit scarring associated with one form will generally be similarly effective in the other form of scarring.

Fibrotic disorders are usually chronic. Examples of fibrotic disorders include cirrhosis of the liver, liver fibrosis, glomerulonephritis, pulmonary fibrosis, chronic obstructive pulmonary disease, scleroderma, myocardial fibrosis, fibrosis following myocardial infarction, central nervous system fibrosis following a stroke, neuro-degenerative disorders (e.g. Alzheimer's Disease, multiple sclerosis), proliferative vitreoretinopathy (PVR), arthritis and adhesions e.g. in the digestive tract, abdomen, pelvis, spine.

If not treated the pathological effects of scarring associated with fibrotic disorders may lead to organ failure, and ultimately to death.

Whilst much of the present specification concentrates primarily on the effects of scarring (whether scarring that results from healing of a wound, or scarring associated with fibrotic disorders) in man, it will be appreciated that many aspects of the scarring response are conserved between most species of animals. Thus, the problems outlined above are also applicable to non-human animals, and particularly veterinary or domestic animals (e.g. horses, cattle, dogs, cats etc). By way of example, it is well known that adhesions resulting from the inappropriate healing of abdominal wounds constitute a major reason for the veterinary destruction of horses (particularly race horses). Similarly the tendons and ligaments of domestic or veterinary animals are also frequently subject to injury, and healing of these injuries may also lead to scarring associated with increased animal mortality.

Although the ill effects of scarring (either resulting from normal or aberrant wound healing, or associated with fibrotic disorders) are well known there remains a lack of effective therapies able to reduce these effects. In the light of this absence it must be recognised that there exists a strongly felt need to provide medicaments and treatments that are able to prevent, reduce or inhibit scar formation, whether resulting from healing of a wound, or associated with fibrotic disorders).

In addition to the disadvantages that may arise from scarring (whether scarring that results from healing of a wound, or scarring associated with fibrotic disorders), wounds themselves are a source of discomfort to those afflicted, and may be associated with, or give rise to, a number of clinical difficulties or complications.

Wounds are painful, even aside from the events associated with their formation, and delays in the healing of wounds may be associated with extended incidences of pain to the sufferer. Wounds can also decrease the mechanical function of the injured area.

The continued presence of open wounds is also associated with many clinical problems, including blood loss and the increased incidence of infection.

In the light of the above, it will be seen that the acceleration of healing of wounds is advantageous for many different reasons. The ability to accelerate wound closure, and thereby reduce wound size and many of the disadvantages associated with wounding, through promotion of wound contraction would provide many advantages in clinical management of wounds.

One way in which wound closure may be accelerated is through the promotion of wound contraction. Contraction is a naturally occurring process involved in the wound healing response, which draws together the margins of wounds, thereby decreasing their area. By promoting such naturally occurring contraction (which may be distinguished from the pathological contraction found in conditions such as hypertrophic scarring), it is possible to accelerate wound closure.

Although the need for medicaments and methods capable of promoting contraction, and thus closure, of wounds is recognised by those skilled in the art, there remains a lack of widely applicable therapies that may be used to achieve this end. Accordingly, there is a well recognised requirement for new, alternative, and more effective, medicaments and methods by which such promotion of wound closure may be attained.

There are a number of adverse effects associated with current regimes used in the management of wounds. These include protracted healing times, which may ultimately lead to the development of chronic wounds. Other undesirable effects relate to the qualities of the replacement tissues or organs that are generated via the healing process.

The absence of a universally accepted method for accelerating the healing of wounds is indicative of the need for novel medicaments and methods by which such acceleration may be effected. It is well recognised that there are failings and disadvantages associated with many of the current therapies available. Even in the case of relatively successful therapies, there is scope for improvement in terms of increased efficacy, or other parameters.

The Farnesoid X Receptor (FXR), also known as the nuclear receptor subfamily 1, group H, member 4 (NR1H4), is a nuclear oxysterol receptor that regulates multiple target genes involved in cholesterol homeostasis.

FXR was first reported by Forman et al (1995) as an orphan nuclear receptor, activated by farnesol. FXR is now known to be a global regulator of bile acid metabolism, repressing the synthesis of bile acid compounds in the liver. Chenodeoxycholic acid and other bile acids are the natural ligands for FXR and like other steroid receptors, when activated, it translocates to the cell nucleus, forms a dimer (in this case a heterodimer with RXR) and binds to hormone response elements on DNA which elicits expression or transrepression of gene products. One of the primary functions of FXR activation is the suppression of cholesterol 7 alpha-hydroxylase (CYP7A1), the rate-limiting enzyme in bile acid synthesis from cholesterol.

FXR has four splice variants identified in the human liver and small intestine. The 2 main variants, FXR-alpha and FXR-beta, encode proteins with different N termini. Further alternative splicing generates FXR-alpha and FXR-beta transcripts with a 12-bp insertion that results in a 4-amino acid insertion near the hinge region of the proteins.

The amino acid sequence of the various splice variants of FXR are shown in Sequence ID Nos. 1, 2, 3, 4 and 5; the corresponding DNA sequences encoding these polypeptides are shown in Sequence ID Nos. 5 (encoding Sequence 1 or 2), 6, 7, 8 and 9.

It has previously been suggested that agonists of FXR activity (i.e. compounds capable of interacting with FXR to increase its activity) may be of use in the restoration of epidermal barrier function. FXR activators stimulate the activities of both β-glucocerebrosidase and steroid sulphatase, two key enzymes of mature stratum corneum function, which are induced during the final stages of barrier ontogenesis. Other prior art documents have suggested that compounds that may encompass both agonists and antagonists of FXR may be used therapeutically in conditions characterised by a perturbed epidermal barrier function, or conditions associated with disturbed differentiation or excess proliferation of the epidermis or mucous membrane.

It is an aim of certain aspects of the present invention to provide medicaments suitable for the prevention and/or reduction and/or inhibition of scarring. It is an aim of further aspects of the present invention to provide methods of treatment suitable for use in the prevention, and/or reduction, and/or inhibition of scarring. It is an aim of certain embodiments of the invention to provide medicaments suitable for the prevention and/or treatment of scarring that results from healing of a wound. It is an aim of certain embodiments of the invention to provide medicaments suitable for the prevention and/or treatment of scarring associated with fibrotic disorders. It is an aim of certain embodiments of the invention to provide methods of treatment suitable for use in the prevention and/or treatment of scarring that results from healing of a wound. It is an aim of further embodiments of the invention to provide methods of treatment suitable for use in the prevention and/or treatment of scarring associated with fibrotic disorders. It is an aim of certain embodiments of the invention to provide medicaments that are suitable for the inhibition of scarring, and also the acceleration of wound healing. It is an aim of certain embodiments of the invention to provide methods of treatment able to inhibit scarring and also to accelerate wound healing. The medicaments and/or methods of the invention may constitute alternatives to those provided by the prior art. It is preferred that medicaments and/or methods of treatment provided by the invention may constitute improvements over the prior art.

According to a first aspect of the present invention there is provided the use of an antagonist of FXR activity in the manufacture of a medicament for the prevention, reduction or inhibition of scarring. It will be appreciated that a medicament manufactured in accordance with the first aspect of the invention should preferably be one that provides a therapeutically effective amount of the selected antagonist of FXR activity. A medicament manufactured in accordance with the first aspect of the invention may preferably be able to promote wound contraction (and thereby accelerate wound closure), in addition to preventing, reducing or inhibiting scarring. This first aspect of the invention also provides an antagonist of FXR activity for use as a medicament for the prevention, reduction or inhibition of scarring.

According to a second aspect of the invention there is provided the use of an antagonist of FXR activity in the manufacture of a medicament for the promotion of wound contraction. It will be appreciated that a medicament manufactured in accordance with the second aspect of the invention should preferably be one that provides a therapeutically effective amount of the selected antagonist of FXR activity. A medicament manufactured in accordance with the second aspect of the invention may preferably be able to prevent, reduce or inhibit scarring, in addition to promoting wound contraction (which may accelerate wound closure). This second aspect of the invention also provides an antagonist of FXR activity for use as a medicament for the promotion of wound contraction.

In a third aspect of the invention there is provided a method of preventing, reducing or inhibiting scarring, the method comprising administering a therapeutically effective amount of an antagonist of FXR activity, to a patient in need of such prevention, reduction or inhibition. The amount of the antagonist of FXR activity administered may preferably also be capable of promoting wound contraction (and thereby accelerating wound closure), in addition to preventing, reducing or inhibiting scarring.

In a fourth aspect of the invention there is provided a method of promoting wound contraction, the method comprising administering a therapeutically effective amount of an antagonist of FXR activity to a patient in need of such promoted wound contraction. The amount of the antagonist of FXR activity administered in accordance with this aspect of the invention may preferably be able to prevent, reduce or inhibit scarring, in addition to promoting wound contraction (which may accelerate wound closure).

The skilled person will appreciate that the methods of treatment of the invention may suitably be practiced using the medicaments of the invention.

The present invention is based, in part, on the inventors' new and surprising finding that antagonists of FXR activity may be used to prevent, reduce or inhibit scarring. The ability to prevent, reduce or inhibit scarring is clearly of clinical benefit due to the damaging physical and psychological effects of scars, effects which are described elsewhere in the specification. There are no previous reports that would lead the skilled person to believe that antagonists of FXR activity may be used to prevent, reduce or inhibit scarring (which, in the example of skin scarring, depends mainly on alterations to the structure of the neodermis, as discussed elsewhere in the specification), and none of the disclosures of the prior art would in any way lead the skilled person to consider that antagonists of FXR activity may be used in this way.

The inventors' have also found that antagonists of FXR activity may be used to promote contraction of wounds. This finding is also new and surprising in the light of the prior art. The ability to promote wound contraction is clinically advantageous since it may reduce the size of wounds. This ability to amplify the natural wound contraction response to bring about therapeutic benefits should be differentiated from pathological contraction that may occur in conditions such as hypertrophic scarring. It will be appreciate that wounds in which contraction has been promoted to bring about a reduced size will be more amenable to procedures intended to bring about wound closure, such as grafting or suturing of wounds. Furthermore, smaller wounds produced on promotion of contraction may heal faster than wounds the contraction of which has not been promoted.

Surprisingly, the inventors have found that promotion of wound contraction achieved by the medicaments and methods of the invention is not accompanied by an increase in re-epithelialisation of treated wounds.

The inventors have found that the same antagonists of FXR activity (and the same amounts of such antagonists) are able to confer both the ability to inhibit scarring and the ability to promote wound contraction. Thus medicaments or methods of the invention for the inhibition of scarring may also be used to promote wound contraction, and medicaments and methods of the invention for the promotion of wound contraction may also be used to inhibit scarring.

In some contexts the ability of the medicaments or methods of the invention to inhibit scarring will be sufficient to make them therapeutically advantageous, and their ability to promote wound contraction may be only an ancillary benefit, or of no therapeutic importance whatsoever. This may, for example, be the case for medicaments or methods of the invention that are to be used in the inhibition of scarring associated with fibrotic disorders where there is no wound contraction of which may be promoted.

There will be other contexts in which the ability of the medicaments or methods of the invention to promote wound contraction will be the determining factor in assessing their therapeutic effectiveness, and their ability to inhibit scarring will be of lesser benefit or no benefit at all.

Skin wounds, the healing of which may be accelerated using the medicaments and methods of the invention, include both chronic wounds and acute wounds. Examples of suitable chronic or acute wounds, the healing of which may be accelerated in accordance with the invention, are set out elsewhere in the specification.

Examples of specific wounds, other than those of the skin, which may benefit from promotion of wound contraction in accordance with the present invention include, but are not limited to, those selected from the group consisting of: gastrointestinal ulcers, lung abscesses and wounds associated with myocardial infarction.

The inventors believe that medicaments or methods of the invention may be used to effectively promote the contraction of various gastrointestinal ulcers, such as peptic, gastric, duodenal and esophageal ulcers. These ulcers may be considered for the purposes of the present invention to constitute chronic wounds.

Lung abscesses may arise as a result of inflamed pleura, which lead to the formation of a pus-filled cavity and the subsequent loss of lung parenchyma. Presently, treatment of lung abscesses tends to involve the use of antibiotics to address the infection, but such treatment may still leave a hole in the lung that is eventually repaired to leave a dense scar. The presence of such a scar can significantly decrease lung function. The inventors believe the medicaments and methods of the invention may be used beneficially to promote wound contraction (and thereby decrease the size of wound and accelerate wound closure) and to inhibit scar formation.

Myocardial infarction can result in coagulative necrosis, which in turn causes the activation of neutrophils to remove the necrotic debris. This process may result in the formation of a hole in the tissue that cannot be replaced by myocardium. The inventors believe that the promotion of wound contraction using the medicaments or methods of the invention may beneficially reduce the size of such a hole and thus aid suture techniques used in repair of the defect, and may inhibit scarring, thereby leading to the formation of a more functionally effective repaired tissue.

The inventors believe that the medicaments or methods of the invention may be used to promote contraction of large open wounds that are subsequently to be subject to grafting or suturing procedures. In these cases the ability to reduce wound area by promoting contraction will be advantageous as a “preparatory treatment” prior to grafting or suturing, since it will reduce the amount of graft material required, or the distance between wound edges to be sutured together. If re-epithelialisation of the wound bed occurs during such preparatory treatment it may interfere with the grafting healing process, and in particular may reduce integration of subsequently grafted material. Accordingly it will be seen that the ability of certain embodiments of the medicaments or methods of the invention to promote wound contraction without re-epithelialisation will be of particular benefit in the treatment of wounds prior to grafting or suturing. In such cases the inhibition of scarring (although desirable) is very much of lesser consideration than the promotion of wound contraction.

Although the promotion of wound contraction may be of benefit in many clinical contexts, there will be certain wounds in which the promotion of contraction will not be beneficial. Typically such wounds may be wounds covering a relatively large surface area, or located over a joint or other articulation (where contraction may, in some circumstances, be associated with limitation of function). Examples of wounds for which it may be preferred not to promote contraction, using the medicaments or methods of the invention, include, but are not limited to, the group consisting of: graft donor sites; burns wounds; sunburn wounds; wounds associated with “skin peels” such as “chemical peels” (such as alphahydroxy acid peels, trichloroacetic acid peels or phenol peels) or laser peels; wounds associated with dermabrasion; wounds associated with dermaplaning; wounds associated with photorefractive keratectomy (PRK); and wounds associated with laser tattoo removal.

Without detracting from the above, it will generally be preferred that medicaments or methods in accordance with the invention are used to both inhibit scarring, and also to promote wound contraction. The medicaments or methods of the invention may be used to inhibit scarring that results from healing of a wound, or, additionally or alternatively, may be used to inhibit scarring associated with a fibrotic disorder.

The inventors believe that the medicaments or methods of the invention may be used to inhibit scarring, and/or to promote wound contraction, throughout the body, as considered elsewhere in the specification. However, it is generally preferred that the medicaments or methods of the invention be used to inhibit scarring of the skin (whether such scarring results from the healing of a wound, or is associated with a fibrotic disorder), and/or to promote the contraction of skin wounds. The medicaments and methods of the invention may preferably be used to both inhibit scarring of skin wounds whilst also promoting contraction of such wounds.

Although skin wounds and scars represent preferred wounds and scars that may be treated using the medicaments and methods of the invention, the inventors believe that the medicaments and methods of the invention may be used to inhibit scarring, and/or promote wound contraction, at a range of sites throughout the body.

By way of example, preferred wounds in respect of which the medicaments or methods of the invention may be used to inhibit scarring, and/or to promote wound contraction, may be selected from the group consisting of: wounds of the skin; wounds of blood vessels; wounds of the peripheral or central nervous system (where inhibition of scarring may enhance neuronal reconnection); wounds of tendons, ligaments or muscle; wounds of the oral cavity, including the lips and palate (such as in wounds associated with the treatment of cleft lip or palate); wounds of the internal organs such as the liver, heart, brain, digestive tissues and reproductive tissues; and wounds of body cavities such as the abdominal cavity, pelvic cavity and thoracic cavity (where inhibition of scarring may reduce the number of incidences of adhesion formation and/or the size of adhesions formed).

The medicaments or methods of the invention may be used to inhibit scarring and/or promote contraction, associated with acute wounds or with chronic wounds. Examples of specific acute wounds and specific chronic wounds that may derive particular benefit from the medicaments and methods of the invention are considered elsewhere in the specification.

The medicaments or methods of the invention may be used to inhibit scarring associated with fibrotic disorders occurring throughout the body. Preferred fibrotic disorders may be selected from the group consisting of: skin fibrosis; scleroderma; progressive systemic fibrosis; lung fibrosis; muscle fibrosis; kidney fibrosis; glomerulosclerosis; glomerulonephritis; uterine fibrosis; renal fibrosis; cirrhosis of the liver, liver fibrosis; chronic obstructive pulmonary disease; fibrosis following myocardial infarction; central nervous system fibrosis, such as fibrosis following stroke; fibrosis associated with neuro-degenerative disorders such as Alzheimer's Disease or multiple sclerosis; fibrosis associated with proliferative vitreoretinopathy (PVR); restenosis; endometriosis; ischemic disease and radiation fibrosis.

Various terms that are used in the present disclosure to describe the invention will now be explained further. The definitions provided below may be expanded on elsewhere in the specification as appropriate, and as the context requires.

### “Antagonist of FXR Activity”

For the purposes of the present invention, an “antagonist of FXR activity” is to be understood to encompass any substance that is capable of reducing the activity of FXR to a therapeutically effective extent. A therapeutically effective antagonist of FXR activity, suitable for use in the medicaments or methods of the invention, is any antagonist of FXR activity that is capable of preventing, reducing or inhibiting scarring, and/or promoting wound contraction.

Guggulsterone (Z), described further below, is an antagonist that is capable of inhibiting scarring, and also capable of promoting wound contraction. Accordingly, guggulsterone (Z) represents a preferred antagonist of FXR activity for use in the medicaments or methods of the invention.

FXR is a ligand-activated nuclear hormone receptor, which contains a ligand-binding domain. Upon ligand binding the conformation of FXR changes allowing interaction of FXR with a co-activator protein; it has been suggested that guggulsterone antagonizes the recruitment of this co-activator, preventing FXR mediated down-stream effects (Wu et al, 2002.

Preferably a therapeutically effective antagonist of FXR activity suitable for use in accordance with the present invention may be one that is capable of inhibiting scarring that may otherwise result from a wound to which the antagonist is provided and/or is capable of promoting contraction of such a wound. Additionally, or alternatively, a therapeutically effective antagonist of FXR activity suitable for use in accordance with the present invention may be one capable of inhibiting scarring associated with a fibrotic disorder to which the antagonist is provided.

Antagonists of FXR activity suitable for use in the medicaments or methods of the invention may include competitive antagonists (which bind to the same receptor binding site as the agonist) and/or non-competitive antagonists (which bind to a different receptor binding site than that bound by the agonist). In addition to such competitive and non-competitive antagonists, it will be appreciated that other substances may be used to reduce FXR activity therapeutically, and thereby inhibit scarring, and that these substances will also constitute suitable antagonists of FXR activity within the context of the present invention. Examples of antagonists of this sort include substances capable of reducing the expression of FXR, and thereby therapeutically reducing FXR activity. Suitable antagonists capable of reducing the expression of FXR include RNA interference (RNAi) agents, antisense nucleic acids, ribozymes, and agents such as aptamers that are capable of binding to nucleic acids encoding FXR to prevent their transcription or translation (it will be appreciated that certain of these agents, such as aptamers, are also able to antagonise the activity of FXR even once this protein has been expressed).

The amino acid sequences of the various splice variants of FXR are shown as Sequence ID Nos. 1, 2, 3 and 4, and the sequences of mRNA molecules encoding these FXR splice variants are shown as Sequence ID Nos. 5, 6, 7 and 8 respectively. Given these sequences, the skilled person will be readily able to devise suitable sequences of oligonucleotides or ribozymes complementary to mRNA encoding FXR. Furthermore, the skilled person may use the information provided in the specification to allow the development of aptamers capable of binding to such mRNAs encoding FXR.

Gene silencing is a process by which double stranded RNA triggers the destruction of mRNAs sharing the same sequence. RNA interference (RNAi) is initiated by the conversion of double stranded RNA into 21-23 nucleotide fragments, termed small interfering RNAs (siRNAs) that direct the degradation of the target RNAs. Typically siRNA selection is based on the selection of three to four, 21 nucleotide sequences in the target mRNA that begin with an AA dinucleotide. These 21 nucleotide sequences should ideally have a 30 to 50% content of nucleotides G or C (since these are found to be more active than those with a higher G/C content), and not contain stretches of more than 4 T's or A's in the target sequence, since a 4-6 nucleotide poly(T) tract acts as a termination signal for the enzyme RNA pol III. There are several methods for preparing siRNAs, once they have been designed, including chemical synthesis, in vitro transcription, siRNA expression vectors, and PCR expression cassettes. RNAi has been shown in vivo to prevent ocular scarring by targeting the TGF-β pathway using siRNAs derived from the coding sequence of the human TGF-beta receptor II (TβRII) gene (Nakamura, et al 2004). The use of these strategies to antagonise the activity of FXR represents a preferred embodiment of the medicaments or methods of the invention.

Antisense oligonucleotides target specific mRNA molecules that are complementary to the sequence of the oligonucleotide. The mRNA:oligonucleotide hybrid molecules are recognized by RNase H, which subsequently degrades the mRNA. Typically the most efficient antisense oligonucleotides are between 15 and 25 nucleotides in length and are complementary to sequence surrounding the initiator AUG site of the mRNA. Other sequence motifs that can be targeted to reduce mRNA levels are regions of the mRNA that interact with proteins, ribosomes, spliceosomes and other large entities. Although theoretically chosen, antisense oligonucleotide sequences may be predicted to bind efficiently to target mRNA. Secondary and tertiary folding can make portions of the mRNA inaccessible to the oligonucleotides, and so, in practice, several different antisense oligonucleotides are normally designed and tested.

Natural phosphodiester oligonucleotides are rapidly degraded in living systems by ubiquitous nucleases. Synthetic oligonucleotide variants have therefore been designed that are nuclease-resistant. Modifications to improve stability include the use of phosphorothioate oligonucleotides and 2′-O-alkyl modified oligonucleotides. Another alternative involves the use of peptide nucleic acid (PNA) technology in which the sugar backbone of the oligonucleotide is swapped for a pseudopeptide. The PNA mimics the behaviour of DNA and binds complementary nucleic acid strands. The neutral backbone of PNA results in stronger binding and greater specificity than normally achieved with DNA oligonucleotides.

Oligonucleotides designed and generated in accordance with the considerations outlined above represent preferred antagonists of FXR activity for use in the medicaments or methods of the invention.

Ribozymes have the ability to catalyse both RNA splicing and cleavage. RNA-cleaving ribozymes gain their target specificity from Watson-Crick base-pairing between the ribozyme's binding-arm sequences and sequences that flank the cleavage site of the target RNA. Once bound, their mechanism of cleavage involves attack of the 2′-OH that is 5′ to the scissile bond in the target, thus destabilizing the target RNAs phosphate backbone. Upon cleavage, the resultant products dissociate from the ribozyme complex and the ribozyme is released and may bind and cleave other targets again. The cleavage event renders the mRNA untranslatable and leads to further degradation of the target by cellular ribonucleases.

Typically, the mRNA target site for ribozymes is about 15 nucleotides in length, but since these ribozymes bind their cognate RNA using two independent binding arms 6-7 nucleotides in length (interrupted by the nucleotide being cleaved), ribozyme binding to the target is weaker than it would be to a contiguous sequence of 13-15 nucleotides. This weak binding leads to better sequence specificity, since it makes the ribozyme more sensitive to the effects of mismatches. To protect against degradation by host nucleases, ribozymes have been developed that contain stabilizing chemical modifications, primarily at the 2′-OH position of the sugar residue in each nucleotide. The inventors believe that ribozymes may constitute suitable antagonists of FXR activity for use in the medicaments or methods of the invention.

Aptamers are nucleic acid molecules that assume a specific, sequence-dependent shape and bind to specific target ligands based on a lock-and-key fit between the aptamer and ligand. Typically, aptamers may comprise either single- or double-stranded DNA molecules (ssDNA or dsDNA) or single-stranded RNA molecules (ssRNA). Aptamers may be used to bind both nucleic acid and non-nucleic acid targets. Preferably aptamers may be used to bind to gene expression products having a molecular weight of between 100 and 10,000 Da. ssDNA aptamers may be preferred when it is wished to bind, and thus neutralise, gene expression products comprising DNA.

Suitable aptamers may be selected from random sequence pools, from which specific aptamers may be identified which bind to the selected target molecules with high affinity. Methods for the production and selection of aptamers having desired specificity are well known to those skilled in the art, and include the SELEX (systematic evolution of ligands by exponential enrichment) process. Briefly, large libraries of oligonucleotides are produced, allowing the isolation of large amounts of functional nucleic acids by an iterative process of in vitro selection and subsequent amplification through polymerase chain reaction.

The use of aptamers for the reduction of FXR expression may be advantageous, since aptamers have relatively stable shelf lives. Aptamers suitable for use in the methods of the invention may preferably be stabilized by chemical modifications (for example 2′—NH₂ and 2′ —F modifications).

Aptamers designed and generated in accordance with these considerations may constitute preferred antagonists of FXR activity suitable for use in the medicaments and methods of the invention.

The skilled person will appreciate that a mixture of two, or more, different antagonists of FXR activity may be used in the medicaments or methods of the invention to inhibit scarring and accelerate wound healing, and such use may represent a preferred embodiment of the invention.

A person skilled in the art may well be able to identify whether or not a substance of interest is an antagonist of FXR activity by reference to known texts, tables or other publications. However, the ability of a substance of interest to antagonise FXR activity (thereby indicating that the substance of interest is suitable for use in accordance with the medicaments and methods of the invention) may readily be determined with reference to well known assays, examples of which are discussed in greater detail elsewhere in the specification. Such assays may be used as the basis for high throughput screening designed to identify suitable antagonists for use in accordance with the invention.

An example of a suitable assay, by which the ability of a compound of interest to antagonise FXR activity may be investigated, is provided by the transient transfection and reporter gene assay described in Nishimaki-Mogami et al, Biochem. Biophys. Res. Comm., 2006. Briefly, HepG2 cells are maintained in DMEM containing 10% FCS and 100 μg/mL kanamycin, and seeded in 24-well plates, 24 hours prior to transfection. The plated cells are then transfected with pFXRE-tk-Luc, pcDNA3.1 expression vectors for human FXR (NR1H4) and RXRα, and either a Renilla luciferase vector or pSV-β-galactosidase vector. Three hours after transfection, cells are exposed for 24 hours to bile acids or bile alcohols after which cells are lysed and luciferase activity determined. In this assay, compounds acting as antagonists would show a dose responsive reduction in luciferase activity. Co-incubation of compounds of interest with known agonists should also show a decrease in luciferase activity.

An antagonist of FXR activity suitable for use in the medicaments or methods of the invention may preferably be one that demonstrates FXR antagonist activity as assessed by the assay set out above.

Guggulsterone (Z) is a preferred example of an antagonist of FXR activity suitable for use in the medicaments or methods of the invention. Guggulsterone (Z) has an empirical formula C₂₁H₂₈O₂, and molecular weight 312.45, and at room temperature is a light yellow solid that is soluble in DMSO (5 mg/mL) or ethanol. It has the systematic name of (17Z)-Pregna-4,17(20)-diene-3,16-dione-4,17(20)-cis-Pregnadiene-3,16-dione.

Guggulsterone (Z) (and its stereoisomer E-guggulsterone) are both antagonist ligands for the bile acid receptor Farnesoid X receptor (FXR). (E) and (Z)-guggulsterone are stereoisomers. The structure of guggulsterone (Z) is shown below.

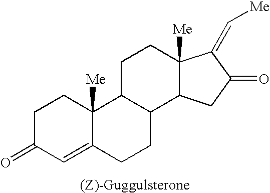

Guggelsterone (Z) is readily available commercially, for example from Calbiochem (under the catalogue number C370690).

A wide range of suitable antagonists of FXR activity, other than guggulsterone (Z), or E-guggulsterone, are known to those skilled in the art. Some examples of such suitable antagonists are set out below.

It has recently been shown that natural products isolated from the marine sponge Spongia sp. inhibit transactivation of FXR (Bioorg. Med. Chem. Lett., 2006, 16, 5398-5402). These compounds belong to a class known as scalaranes. Representative structures of these compounds are shown below, and these compounds are believed to inhibit FXR transactivation with around the same IC₅₀ values as guggulsterone; thereby making them suitable antagonists of FXR activity for use in the medicaments or methods of the invention.

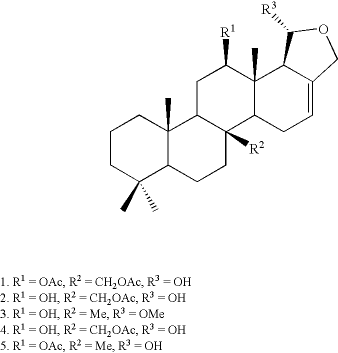

A further antagonist of FXR activity that may be used in the medicaments or methods of the invention is a compound referred to as 80-574 (for example in Pellicciari et al., J. Med. Chem., 2005 48, 6948-6955). The structure of this compound is shown below.

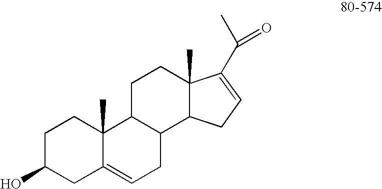

It has also been reported that a series of 5α-bile alcohols act as FXR antagonists (Nishikami-Mogami et al., Biochem. Biophys. Res. Comm., 2006, 339, 386-391). It is important to note that it is only the α-configuration of the bile alcohols that provide antagonist activity, whereas the β-alcohols are agonists. Accordingly α-bile alcohols of this type, and particularly the examples suggested below, represent antagonists of FXR activity suitable for use in the medicaments or methods of the invention. This change in configuration between α and β-bile alcohols could be a key determining factor as to which bile alcohols are antagonists or agonists. This could be further investigated by testing of additional examples of these compound classes in an assay such as mentioned elsewhere in the specification to build a structure activity relationship.

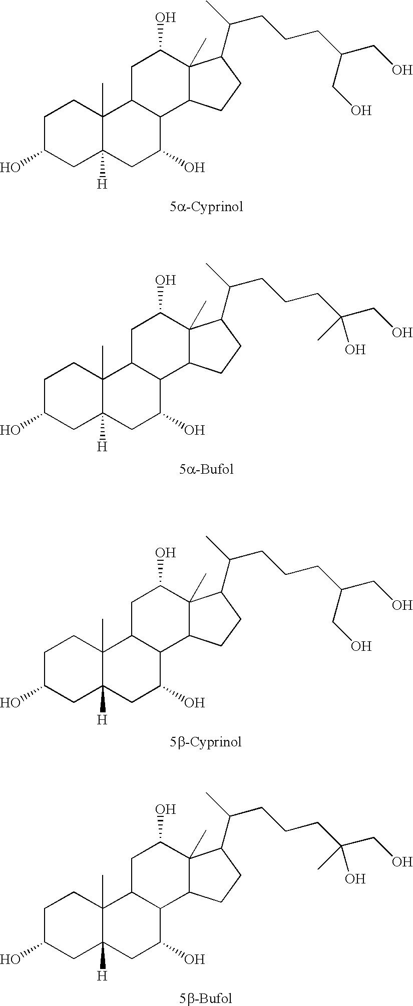

It will be appreciated that the group of antagonists of FXR activity suggested above are provided by way of example only, and that other suitable antagonists not listed here may also be used in the medicaments or methods of the invention.

### “Medicaments of the Invention”

Except where the context requires otherwise, references to “medicaments of the invention” should generally be taken as referring to medicaments prepared in accordance with any suitable aspect of the invention. Medicaments of the invention will generally comprise a pharmaceutically acceptable excipient, diluent or carrier in addition to the antagonist of FXR activity.

Medicaments of the invention may preferably be suitable for topical administration of an antagonist of FXR activity. Medicaments of the invention may preferably be in the form of an injectable solution comprising an antagonist of FXR activity. Solutions suitable for localised injection (such as intradermal injection) constitute particularly preferred forms of the medicaments of the invention.

An antagonist of FXR activity may preferably be administered either to an existing scar, or to a site where scarring may be expected to occur. For example, an antagonist of FXR activity may be administered to a patient's wound that would otherwise be likely to give rise to a scar. An antagonist of FXR activity may be administered to an existing scar to prevent the further progression of scarring. Administration of antagonists of FXR activity to an existing scar may also reduce the level of scarring associated with the existing scar. It will thus be appreciated that an antagonist of FXR activity may be administered to a site of a fibrotic disorder in order to prevent further scarring, and/or to reduce scarring that has already occurred associated with the fibrotic disorder. Preferred routes of administration that may be used in accordance with all of the embodiments considered above include topical administration, and particularly topical injection (such as intradermal injection) of suitable antagonists.

### “Therapeutically Effective Amount”

For the purposes of the present invention a “therapeutically effective amount” of an antagonist of FXR activity is considered to be an amount of such an antagonist that is able to inhibit scarring, and/or to promote wound contraction. The scarring that is to be inhibited may be scarring that results from healing of a wound, and/or scarring associated with a fibrotic disorder.

A preferred therapeutically effective amount of an antagonist of FXR activity may be an amount that is able to bring about a required inhibition of scarring, and that is also able to bring about a required promotion of wound contraction.

In the context of the inhibition of scarring, a therapeutically effective amount of an antagonist of FXR activity may be an amount that is effective to reduce scarring by at least 10% compared to scarring that would otherwise be expected to occur without administration of the antagonist. Preferably a therapeutically effective amount may be capable of achieving at least a 20% reduction in scarring, more preferably at least 50%, even more preferably at least 75% and most preferably at least a 90% reduction in scarring, compared to scarring that would otherwise be expected to occur without administration of the antagonist.

In the case of scarring that results from healing of a wound, a therapeutically effective amount of an antagonist of FXR activity may be an amount that is effective to reduce scarring of a treated wound by at least 10% compared to scarring produced on healing of a comparable untreated wound. “Treated wounds” and “untreated wounds” are defined further elsewhere in the specification. Preferably a therapeutically effective amount may be capable of achieving at least a 20% reduction in scarring, more preferably at least 50%, even more preferably at least 75% and most preferably at least a 90% reduction in scarring compared to scarring produced on healing of an untreated wound.

In the case of scarring that is associated with a fibrotic disorder, a therapeutically effective amount of an antagonist of FXR activity may be an amount that is effective to reduce scarring of a treated site of fibrosis by at least 10% compared to the amount scarring that would otherwise be present at a comparable untreated site of fibrosis. A “treated site of fibrosis” and “untreated site of fibrosis” are defined further elsewhere in the specification. Preferably a therapeutically effective amount may be capable of achieving at least a 20% reduction in scarring, more preferably at least 50%, even more preferably at least 75% and most preferably at least a 90% reduction in scarring compared to scarring present at a comparable untreated site of fibrosis.

A therapeutically effective amount of an antagonist of FXR activity for use in the inhibition of scarring may preferably be an amount able to therapeutically alter the abundance and/or orientation of ECM components (such as collagen) in a treated scar.

In the context of promotion of wound contraction, a therapeutically effective amount of an antagonist of FXR activity may be an amount that is effective to lead to a treated wound contracting at a rate at least 5% faster than an untreated wound, preferably at a rate at least 10% faster, more preferably at least 15%, 20% or 25% faster; yet more preferably at least 50% faster, still more preferably at least 75% faster, and most preferably 100% (or more) faster. Suitable methods by which contraction of wounds may be quantified in order to assess promotion of wound contraction are described elsewhere in the specification.

A medicament of the invention should provide a therapeutically effective amount of an antagonist of FXR activity. Preferably a medicament of the invention may be provided in the form of one or more dosage units, each dosage unit comprising a known multiple or fraction of a therapeutically effective amount of an antagonist of FXR activity.

The skilled person will appreciate that an antagonist of FXR activity that has little inherent therapeutic activity will still be therapeutically effective if administered in a quantity that provides a therapeutically effective amount.

The inventors have found that antagonists of FXR activity are able to inhibit scarring at all concentrations thus far investigated. Inhibition of scarring caused by the medicaments or methods of the invention may be assessed microscopically or macroscopically, as described further below.

The inventors have also found that all concentrations of antagonists of FXR activity tested thus far are able to promote wound contraction.

It will be appreciated that an amount of an antagonist of FXR activity that is able neither to inhibit scarring (or an amount that actually serves to worsen scarring), nor to promote wound contraction, will not constitute a therapeutically effective amount for the purposes of the present invention.

It may be preferred that medicaments of the invention be used in an administration pattern comprising administering a therapeutically effective amount of the antagonist of FXR activity prior to formation of a wound, and administering a further therapeutically effective amount of the antagonist of FXR activity after formation of the wound. The further therapeutically effective amount of the antagonist of FXR activity may preferably occur approximately 24 hours after formation of the wound.

The use recited above may further comprise the repetition of administration of therapeutically effective amounts of an antagonist of FXR activity for as long as necessary to achieve prevention, reduction or inhibition of scarring, and/or to promote wound contraction.

### “Treated Wounds”, “Control-Treated Wounds” and “Untreated Wounds”

A “treated wound” in the context of the present disclosure is any wound that has been provided with a therapeutically effective amount of a medicament of the invention, or a therapeutically effective amount of an antagonist of FXR activity administered in accordance with a method of treatment of the invention.

“Control-treated wounds” and “untreated wounds” in the present context are respectively wounds treated with a relevant control, and wounds that have not been treated before, or during, healing. Control wounds will not be treated with a medicament of the invention, and preferably will not be treated with a therapeutically effective amount of an active compound that inhibits scarring and/or promotes wound contraction. That said, wounds treated with medicaments known from the prior art may constitute suitable control wounds for comparative purposes (for example to illustrate increased efficiency or effectiveness of medicaments of the invention as compared to those already known). A “diluent control-treated wound” will be an untreated wound to which a control diluent has been administered, and a “naïve control” will be an untreated wound made without administration of an antagonist of FXR activity, or a suitable control diluent, and left to heal without therapeutic intervention.

### Treated and Untreated Sites of Fibrosis

A treated site of fibrosis in the context of the present disclosure is a site of fibrosis to which has been provided a therapeutically effective amount of an antagonist of FXR activity. Accordingly, an untreated site of fibrosis is a site of fibrosis that has not been provided with a therapeutically effective amount of an antagonist of FXR activity.

### “Treated Scar”

Treatment of wounds with a therapeutically effective antagonist of FXR activity may result in the inhibition of scarring that may otherwise be expected to occur on healing of untreated wounds. The inventors believe that treatment in this manner may have an impact on both the macroscopic and microscopic appearance of scars formed from treated wounds; macroscopically the scars may be less noticeable and blend better with the surrounding normal tissue, microscopically the scars may exhibit an internal structure more akin to that found in normal unwounded tissue (indicative that the scar may have physical or mechanical properties that more closely resemble those of unscarred tissue). For example, in the case of scars that result from the healing of skin wounds, a treated scar may, when viewed microscopically, exhibit an abundance and orientation of ECM molecules such as collagen that is more similar to that found in normal skin than that found in untreated scars.

Treatment of fibrotic disorders with a therapeutically effective antagonist of FXR activity may result in the inhibition of scarring that may otherwise be expected to be associated with the fibrotic disorder. The inventors believe that treatment in this manner may have an impact on both the macroscopic and microscopic appearance of scars associated with fibrotic disorders, such that the macroscopic and/or microscopic structure of a scar at a treated site of fibrosis will be more akin to that found in normal non-fibrotic tissue. For example, in the case of fibrosis involving the skin, a treated scar may, when viewed microscopically, exhibit an abundance and orientation of ECM molecules, such as collagen, that is more similar to that found in normal skin than that found in untreated scars.

For the present purposes a “treated scar” should be taken to encompass:
i) a scar that results from healing of a treated wound (i.e. a wound treated with a therapeutically effective amount of an antagonist of FXR activity); and/or
ii) a scar produced at a site of a fibrotic disorder that has been treated with a therapeutically effective amount of an antagonist of FXR activity; and/or
iii) a scar to which a therapeutically effective amount of an antagonist of FXR activity has been administered.

### “Untreated Scar”

By way of contrast, an “untreated scar” should be taken to encompass:
i) a scar that results from healing of an untreated wound (for example a wound treated with a placebo, control, or standard care); and/or
ii) a scar (whether resulting from a wound, or associated with a fibrotic disorder) to which a therapeutically effective amount of an antagonist of FXR activity has not been administered.

Untreated scars may typically be used as comparators in assessing the inhibition of scarring that may be evident in a treated scar. Suitable comparator untreated scars of this type may preferably be matched to the treated scar with reference to one or more criteria selected from the group consisting of: scar age; scar size; scar site; patient age; patient race and patient gender.

### Inhibition of Scarring

The prevention, reduction or inhibition of scarring within the context of the present invention should be understood to encompass any degree of prevention, reduction or inhibition of scarring associated with a treated scar, as compared to the level of scarring in an untreated scar.

The inventors believe that the anti-scarring activity (“inhibition of scarring” or “inhibiting scarring”) that may be achieved using the medicaments or methods of the invention may be used in the treatment of existing scars to reduce the scarring present. Furthermore, the inventors believe that the anti-scarring activity of the medicaments or methods of the invention may be used to prevent scar formation occurring. This may particularly be the case when the medicaments or methods of the invention are used prophylactically, either before wound formation, or before the onset of scarring associated with a fibrotic disorder. Throughout the specification references to “prevention”, “reduction” or “inhibition” of scarring are generally to be taken, except where the context requires otherwise, to represent effectively equivalent activities, mediated by equivalent mechanisms relating to the antagonism of FXR activity, and that are all manifested in anti-scarring activity.

It may be preferred that inhibition of scarring achieved using the medicaments or methods of the invention should be sufficient to reduce scarring by at least 10% compared to scarring that would otherwise be expected to occur without administration of the antagonist. Preferably inhibition of scarring achieved using the medicaments or methods of the invention may bring about at least a 20% reduction in scarring, more preferably at least 50%, even more preferably at least 75% and most preferably at least a 90% reduction in scarring compared to scarring that would otherwise be expected to occur without administration of an antagonist of FXR activity.

The inhibition of scarring that may be achieved utilising therapeutically effective amounts of an antagonist of FXR activity may be of benefit in almost all circumstances where unwanted scarring would otherwise occur.

The following paragraphs are in no way intended to limit the uses to which methods and medicaments of the invention may be put, but may provide useful guidance as to contexts in which it may be wished to inhibit scar formation by use of a therapeutically effective amount of an antagonist of FXR activity. It will be appreciated that in many of the contexts considered below it may also be desirable to promote wound contraction using the medicaments or methods of the invention.

The use of methods and medicaments of the invention to inhibit scarring may bring about a notable improvement in the cosmetic appearance of an injured area thus treated. Cosmetic considerations are important in a number of clinical contexts, particularly when scars may be formed at prominent body sites such as the face, neck and hands. Consequently it is a further preferred embodiment that the medicaments and methods of the invention be used to inhibit scarring at sites where it is desired to improve the cosmetic appearance of a scar formed. Indeed, it is a preferred embodiment that the medicaments and methods of the invention be used to inhibit scarring associated with cosmetic surgery. Since the great majority of cosmetic surgeries consist of elective surgical procedures it is readily possible to administer a therapeutically effective amount of an antagonist of FXR activity prior to surgery, and/or immediately following closure of the wound (e.g. with sutures), and this use represents a particularly preferred embodiment of the invention.

The medicaments of the invention may be used to inhibit scarring associated with surgical procedures and/or to promote contraction of wounds associated with surgical procedures. When used in association with surgical procedures a preferred route by which an antagonist of FXR activity may be administered is via intradermal injection. Such injections may form raised blebs, which may then be incised as part of the surgical procedure, or alternatively the bleb may be raised by injecting the wound margins after the wound has been closed e.g. by sutures.

The cosmetic outcome of surgical procedures is also an important consideration in plastic surgery, and the use of methods or medicaments of the invention to inhibit scarring associated with plastic surgery constitutes a further preferred embodiment of the invention. The medicaments or methods of the invention may also be used to promote contraction of wounds associated with plastic surgery. The inventors believe that the use of the medicaments or methods of the invention in this embodiment may be beneficial both in reducing the size of such wounds (via promotion of wound contraction), and also in inhibiting scarring that may otherwise occur.

In addition to its cosmetic impact scarring of the skin is responsible for a number of deleterious effects afflicting those suffering from such scarring. For example, scarring of the skin may be associated with reduction of physical and mechanical function, particularly in the case of contractile scars (such as hypertrophic scars) and/or situations in which scars are formed across joints. In these cases the altered mechanical properties of scarred skin, as opposed to unscarred skin, and the effects of scar contraction may lead to dramatically restricted movement of a joint (articulation) so effected. Accordingly, it is a preferred embodiment that suitable medicaments and methods of the invention be used to inhibit scarring covering joints of the body (whether such scars result from the healing of wounds covering the joint, or are associated with fibrotic disorders covering the joint). In another preferred embodiment suitable medicaments and methods of the invention may be used to inhibit scarring at increased risk of forming a contractile scar (in the case of scarring that results from the healing of wounds, this may particularly include wounds of children).

The extent of scar formation, and hence extent of cosmetic or other impairment that may be caused by the scar, may also be influenced by factors such as the tension of the site at which the scar is formed (and in the case of scarring that results from the healing of a wound, the tension at the site where the wound is formed). For example, it is known that skin under relatively high tension (such as that extending over the chest, or associated with lines of tension) may be prone to formation of more severe scars than at other body sites. Thus in a preferred embodiment suitable medicaments and methods of the invention may be used to inhibit scarring at sites of high skin tension. The medicaments and methods of the invention may, for example, be used to inhibit scarring that result from wounds located at sites of high skin tension.

There are many surgical procedures that may be used in scar revision to allow realignment of wounds and scars such that they are subject to reduced tension. In a preferred embodiment the medicaments and methods of the invention may be used to inhibit scarring of wounds during surgical revision of scars. It will be appreciated that medicaments or methods of the invention may also be used to promote contraction of wounds associated with surgical revision of scars, since this will reduce wound size and hence may reduce the time required for healing of such wounds.

Pathological scarring may have more pronounced deleterious effects than arise even as a result of relatively severe normal scarring. Common examples of pathological scars include keloids, hypertrophic scars and pterygium.

Keloid scars (or keloids) constitute a notable example of pathological scarring, and are raised scars that spread beyond the margins of the original wound and invade the surrounding normal skin. Keloids continue to grow over time, do not regress spontaneously, and frequently recur following surgical excision. Keloid scars occur with equal frequency in men and women, mainly from ages 10 to 30, and can result from piercing, surgery, vaccination, tattoos, bites, blunt trauma and burns. A number of studies have suggested that there is an underlying genetic predisposition to keloid formation since keloid scars are more prevalent in dark skinned races, and in individuals of the African Continental Ancestry Group or Asian Continental Ancestry Group.

Keloids appear as elevated scars that may typically be hyperpigmented or hypopigmented in relation to the surrounding skin. Keloids may be characterised on the basis of their tendency to grow beyond the initial boundaries of the wound from which they result. At a microscopic level, keloids may be characterised by the presence of large whorls of collagen, and the predominantly acellular nature of the interior of the lesion.

Hypertrophic scars are raised scars which may have an appearance very similar to keloid lesions. Unlike keloids, hypertrophic scars do not expand beyond the boundaries of the original injury and are not prone to recurrence after excision. Hypertrophic scars may frequently undergo contraction, and it is believed that the contractile nature of hypertrophic scars may be associated with the elevated numbers of myofibroblasts that are frequently reported within these types of scars. Hypertrophic scars may commonly arise as a result of burn or scald injuries, and are particularly common amongst children.

Pterygium is a hypertrophied outgrowth of the subconjunctival tissue to the border of the cornea or beyond. The outgrowth is typically triangular in shape, with the apex pointing towards the pupil. Pterygium may interfere with vision, and may require surgery to remove the hypertrophied tissue. Furthermore, the tissue may frequently re-grow after excision, in the same manner as keloid scars, thus requiring multiple incidences of surgery.

It is recognised that certain types of wound, or certain individuals may be predisposed to pathological scar formation. For instance individuals of the African Continental Ancestry Group or Asian Continental Ancestry Group, or those having a familial history of pathological scarring may be considered to be at increased risk of hypertrophic scar or keloid formation. Wounds of children, and particularly burns wounds of children, are also associated with increased hypertrophic scar formation. Incidences of pterygium may be increased amongst those in whom the eye is frequently exposed to intense sunlight or dust. Accordingly it is a preferred embodiment of the invention that suitable medicaments and methods be used to inhibit scarring of wounds in which there is an increased risk of pathological scar formation.

Although individuals already subject to pathological scarring may suffer from a predisposition to further excessive scar formation, it is often clinically necessary to surgically revise hypertrophic scars or keloids, with an attendant risk of consequential pathological scar formation. Thus, it is a further preferred embodiment of the invention that the medicaments or methods herein described be used to inhibit scarring that results from wounds produced by surgical revision of pathological scars.

The medicaments and methods of the invention may be used to inhibit scarring and/or promote contraction associated with a wide range of wound types which may occur at a wide range of body sites. The medicaments and methods of the invention may be used to inhibit scarring and/or promote contraction associated with wounds selected from the group consisting of: abrasions; avulsions; crush wounds; incisional wounds; lacerations; punctures; and missile wounds. All of these different types of wounds may be suffered by the skin, among other tissues or organs, and all may, to a greater or lesser extent, give result in scarring.

Abrasions are also commonly referred to as “scrapes”. Abrasions occur as a result of the skin being rubbed away by friction against another rough surface. Common examples of abrasions include rope burns and skinned knees. An abrasion may macroscopically appear as lines of scraped skin, possibly including tiny spots of bleeding. Although abrasions represent relatively “shallow” injuries they may give rise to scars, and frequently cover a relatively large area. Accordingly the medicaments and methods of the invention may advantageously be used in the inhibition of scarring resulting from abrasion wounds. It may also be preferred that the medicaments or methods of the invention be used to accelerate the healing of abrasion wounds.

Avulsions occur when an entire bodily structure, or a part of such a structure, is forcibly pulled away from its site. Examples of avulsions include the loss of a permanent tooth or an ear lobe. Avulsions may, for example, arise as a result of explosions, gunshots, and animal bites. An avulsion may characteristically exhibit heavy, rapid bleeding, as well as a noticeable absence of tissue. It will be appreciated that the trauma associated with avulsion injuries may generally lead to extensive scarring, and so it will be appreciated that the medicaments and methods of the invention may advantageously be used in the inhibition of scarring resulting from avulsion wounds. It may also be preferred that the medicaments or methods of the invention be used to promote contraction of avulsion wounds.

Crush wounds typically occur as a result of a heavy object falling onto an individual (or part of an individual). The force thus generated may split the skin and shatter or tear underlying structures. A crush wound may have irregular margins, similar in appearance to those of a laceration; however, the wound will generally be deeper and trauma to underlying muscle and bone may be apparent. As described elsewhere in the specification, the inventors believe that the medicaments and methods of the invention may advantageously be used in the inhibition of scarring that results from both penetrative and non-penetrative injuries, and accordingly the medicaments and methods of the invention may be beneficial in inhibiting scarring resulting from crush wounds. It may also be preferred that the medicaments or methods of the invention be used to promote contraction of crush wounds.

Incisional wounds are also commonly referred to as “cuts”. Incisional wounds result from incision, or slicing, of a tissue with a sharp instrument, which results in a wound with relatively even edges. Incisional wounds can vary greatly in their severity, from minimal wounds (such as a paper cut) to significant wounds such as those arising as a result of surgical incision. An incisional wound may have little or profuse bleeding depending on the depth and length of the wound, and also on the tissue involved. The even edges of incisional wounds will generally readily line up, which may facilitate closure of such wounds. Incisional wounds are a frequent cause of scarring, and it will be appreciated that the medicaments and methods of the invention may advantageously be used in the inhibition of scarring resulting from incisional wounds. It may also be preferred that the medicaments or methods of the invention be used to promote contraction of incisional wounds. Medicaments and methods of the invention may be particularly helpful in aiding the surgical closure of wounds (and in particular the surgical closure of incisional wounds), since they are able to promote contraction of such wounds, and thus reduce the width of treated wounds, thereby aiding their closure.

Lacerations are also frequently referred to as “tears”. These wounds arise as a result of forcible separation of a tissue or organ, which will normally produce a wound having characteristic ragged edges. Lacerations are generally produced by the action of great mechanical forces against the body, either from an internal source as in childbirth, or from an external source like a punch. The laceration arises when the force exerted on a tissue or organ becomes too great for the tissue or organ to bear. A laceration may exhibit little or profuse bleeding, in much the same manner as an incisional wound. In contrast to incisional wounds however, the tissue damage is generally greater and the wound's ragged edges do not line up so readily. This frequent misalignment of the margins of lacerations may contribute to increase scarring resulting from the healing of such wounds, however, the inventors believe that the medicaments and methods of the invention may advantageously be used in the inhibition of scarring resulting from laceration wounds. It may also be preferred that the medicaments or methods of the invention be used to promote contraction of laceration wounds.

Punctures are deep, narrow wounds. Punctures may typically be produced by sharp objects such as nails, knives, and broken glass being driven into the body. The depth of a puncture wound will generally be greater than its length. As a consequence there is generally little bleeding around the outside of the wound although more bleeding may occur inside the wound. This may lead to discoloration around the puncture wound. Although punctures may typically involve only a relatively small surface area of the body, their depth means that a number of different tissue types may frequently be subject to scarring as a result of puncture wounds. Given the ability of the medicaments and methods of the invention to prevent scarring in various different tissues, it will be appreciated that such medicaments and methods may advantageously be used in the inhibition of scarring resulting from puncture wounds. It may also be preferred that the medicaments or methods of the invention be used to promote contraction of puncture wounds.

Missile wounds are also known as “velocity wounds”. Missile wounds are caused by an object entering the body at a high speed, typically a bullet. A missile entry wound may be accompanied by an exit wound, and bleeding may be profuse, depending on the nature of the injury. The inventors believe that the medicaments and methods of the invention may advantageously be used in the inhibition of scarring resulting from missile wounds. It may also be preferred that the medicaments or methods of the invention be used to promote contraction of missile wounds.

Incisional or excisional wounds associated with surgery constitute preferred wounds scarring resulting from which may be inhibited by the medicaments and methods of the invention. Surgical incisional wounds may constitute a particularly preferred group of wounds in respect of which scarring may be inhibited utilising the medicaments and methods of the invention. It may also be preferred that the medicaments or methods of the invention be used to promote contraction of incisional or excisional surgical wounds, thereby reducing their size and facilitating their closure.

It will be appreciated that tissues other than the skin, such as the cornea, may also be subject to wounds of the type described above and elsewhere in the specification. The medicaments and methods of the invention may also be of benefit in inhibiting scarring associated with such wounds in these tissues.

The healing of wounds involving the peritoneum (the epithelial covering of the internal organs, and/or the interior of the body cavity) may frequently give rise to adhesions. Such adhesions are formed by bands of fibrous scar tissue, and can connect the loops of the intestines to each other, or the intestines to other abdominal organs, or the intestines to the abdominal wall. Adhesions can pull sections of the intestines out of place and may block passage of food. Adhesions are also a common sequitur of surgery involving gynaecological tissues. Incidences of adhesion formation may be increased in wounds that are subject to infection (such as bacterial infection) or exposure to radiation. The inventors believe that the ability of the medicaments and methods of the invention to inhibit scarring may reduce the occurrence of adhesions. Accordingly, the use of medicaments or methods of the invention to prevent the formation of intestinal or gynaecological adhesions represents a preferred embodiment of the invention. The medicaments and methods of the invention may also be useful in the inhibition of scarring, including formation of adhesions, that may occur on healing of infected wounds or wounds exposed to radiation. Indeed, the skilled person will appreciate that the use of medicaments or methods of the invention in the inhibition of any scarring involving the peritoneum is a preferred embodiment. Medicaments for this purpose may be administered by lavage, or in a parenteral gel/instillate or locally e.g. from films or carriers inserted at the time of surgery.

The medicaments or methods of the invention are suitable for use in the inhibition of scarring in the eye, and their use in this context represents a preferred embodiment of the invention. The inventors believe that the medicaments or methods of the invention may be used to inhibit scarring that results from healing of wounds to the eye, and/or to inhibit scarring associated with fibrotic disorders of the eye.

In the case of corneal scarring application of the medicament may be by means of local eye drops, sponge applicator, or the like. Corneal scars may result from the healing of corneal wounds such as those produced by LASIK or PRK procedures. Corneal scarring may be assessed by measuring the opacity, or transmitting/refractory properties, of the cornea. Such assessments may, for example, be made using in vivo confocal microscopy.

Scarring elsewhere in the eye, such as at sites of pressure relieving blebs formed in glaucoma surgery, or scarring of the retina associated with proliferative vitreoretinopathy may also be inhibited by the medicaments and methods of the present invention. A therapeutically effective amount of an antagonist of FXR activity may be delivered locally, for example by means of a device implanted in the eye, or by injection.

Scarring in the nervous tissue (either peripheral or central) may be inhibited by the medicaments of the invention. Such scarring may arise as a result of surgery or trauma and may additionally be assessed by future assays of nerve function e.g. sensory or motor tests. Inhibitors of scarring should improve such future outcomes.

Scarring in the blood vessels e.g. following anastomotic surgery, can lead to myointimal hyperplasia and reduction in the volume of the blood vessel lumen (restenosis). This can be measured directly e.g. using ultrasound, or indirectly by means of blood flow. Inhibition of scarring achieved using the medicaments or methods of the invention may lead to a reduction in narrowing of the blood vessel lumen and allow a more normal blood flow. A therapeutically effective amount of an antagonist of FXR activity may be provided to blood vessels by any suitable means. Merely by way of example, these may include direct injection into the walls of the blood vessel before suturing, bathing an anastomotic site in a medium comprising the antagonist of FXR activity, or administration of the antagonist by local applied devices, e.g. stents. Effective inhibition of scarring in blood vessels may be indicated by the maintenance of a normal level of blood flow following blood vessel injury. The medicaments and methods of the invention may also be used to promote contraction of wounds of blood vessels, thereby reducing the size of such wounds and aiding their subsequent closure.

The medicaments or methods of the invention may be used to inhibit scarring in tendons and ligaments. Such scarring may otherwise be expected to occur following surgery or trauma involving tissues of this type. Successful inhibition of scarring may be indicated by restoration of function of tissues treated with the medicaments or methods of the invention. Suitable indicia of function may include the ability of the tendon or ligament to bear weight, stretch, flex, etc.

### “Promoting Contraction of Wounds”

“Promoting contraction of wounds”, or “promoting wound contraction” in the context of the present disclosure should be taken to encompass any increase in the rate at which a treated wound contracts.

Promotion of wound contraction can facilitate closure of wounds either without further intervention (since a wound of reduced size will generally heal faster than a wound of larger size) or by aiding the grafting, suturing or other artificial closure of such a wound. Promotion of contraction of a wound achieved using the medicaments or methods of the invention may preferably lead to a treated wound contracting at a rate at least 5% faster than an untreated or control wound, preferably at a rate at least 10% faster, more preferably at least 15%, 20% or 25% faster; yet more preferably at least 50% faster, still more preferably at least 75% faster, and most preferably 100% (or more) faster. Suitable methods by which promotion of wound contraction may be quantified to assess improvements in the rate of healing are described elsewhere in the specification. In general, suitable assessments of increases in wound contraction may be determined with reference to wound area (assessed microscopically or macroscopically, for instance by means of image analysis).

One measurement that may be used in assessing the rate of contraction of a wound is the rate at which the width of a wound decreases. Promotion of contraction of a wound achieved using the medicaments or methods of the invention may preferably lead to a treated wound in which wound width decreases at a rate at least 5% faster than an untreated wound, preferably at a rate at least 10% faster, more preferably at least 15%, 20% or 25% faster; yet more preferably at least 50% faster, still more preferably at least 75% faster, and most preferably 100% (or more) faster. Suitable methods by which wound width may be measured in order to assess promotion of contraction of wounds are described elsewhere in the specification.

Promotion of contraction using the medicaments or methods of the invention may also give rise to a treated wound having an increased “healing age” when compared with an untreated or control treated wound. Such an increase in healing age may be assessed macroscopically, visually or clinically to determine maturity of the treated wound compared to a suitable untreated or control wound. A wound treated with a therapeutically effective amount of an antagonist of FXR activity in accordance with the present disclosure may preferably lead to a treated wound having a “healing age” that is at least a day faster than an untreated wound, preferably at a rate at least five days faster, more preferably at least ten days faster; yet more preferably at least eleven, twelve, thirteen, fourteen of fifteen days faster, still more preferably fifteen or more days faster, and most preferably 20 (or more) days faster.

As noted elsewhere, the skin suffers from more direct, frequent, and damaging encounters with the external environment than any other organ in the body. As a result the skin suffers from more wounds than other organs, and it is therefore highly desirable to be able to promote contraction of skin wounds in order that this organ may be returned as rapidly as possible to its maximum functional effectiveness. Promoting contraction of skin wounds is thus a preferred use of the medicaments or methods of the present invention.

It will be appreciated that promoting contraction of wounds using the medicaments and methods of the invention may be of particular benefit in cases in which the wound healing response is impaired, inhibited, retarded or otherwise defective as compared to the normal rate of healing. The methods and medicaments of the invention may also be used to promote contraction of wounds in patients that are not subject to an impaired healing response. Illustrative examples of both contexts are set out below.

There are many contexts in which the body's healing response is defective and may benefit from the ability of medicaments or methods of the invention to promote wound contraction. These include conditions such as pemphigus, Hailey-Hailey disease (familial benign pemphigus), toxic epidermal necrolysis (TEN)/Lyell's syndrome, epidermolysis bullosa, cutaneous leishmaniasis and actinic keratosis.

Healing of wounds may also be retarded as a result of the actions of pathogens (such as bacteria, fungi or viruses), chemical insults (such as chemical burns caused by caustic agents, or through the effect of cytotoxic drugs such as those employed in chemotherapy), or as a result of radiation damage (either through particulate radiation or electromagnetic radiation such as gamma radiation, ultraviolet radiation, or the like) such as that occurring in sunburn. Accordingly wounds subject to any of these influences may benefit from promotion of contraction using the medicaments or methods of the invention, since this may help facilitate the closure of such wounds.

It is well known that dermal injuries in the aged heal more slowly than do those of younger individuals. The aged may therefore particularly benefit from promotion of wound contraction using the medicaments and methods of the invention. There are also many other conditions or disorders that are associated with a delayed or otherwise impaired wound healing response. For example patients with diabetes, patients with polypharmacy (for example as a result of old age), post-menopausal women, patients susceptible to pressure injuries (for example paraplegics), patients with venous disease, clinically obese patients, patients receiving chemotherapy, patients receiving radiotherapy, patients receiving steroid treatment or immuno-compromised patients may all suffer from impaired wound healing. In some cases the slower healing response exhibited by such patients may contribute to the development of infections at the site of wounds. The slow wound healing response may also be associated with the formation of chronic wounds, as considered below. Accordingly, it will be appreciated that such patients represent a preferred group that may benefit from use of the methods or medicaments of the invention to promote wound contraction.

Without detracting from the above, it may generally be preferred that the medicaments or methods of the invention may be utilised to promote contraction of wounds of patients not subject to delayed wound healing. Wound contraction promoted in this way will aid the closure of such wounds (whether natural or artificial closure).

The skilled person will immediately appreciate that there is a great benefit to be gained by society from the development of therapeutic agents and techniques that can hasten the healing of otherwise healthy patients. As well as the various benefits considered elsewhere in the specification, promoting wound contraction in this manner can help reduce time spent in convalescence, and can thus benefit productivity. Accordingly, promoting the contraction of wounds of healthy patients is a preferred embodiment of the present invention.

The medicaments and methods of the invention may be used to promote contraction of both chronic wounds and acute wounds. For the purposes of the present invention, a chronic wound may be defined as any wound that does not show any healing tendency within eight weeks of formation when subject to appropriate (conventional) therapeutic treatment. Acute wounds may be any wound other than a chronic wound.

Promoting contraction of chronic wounds is a preferred embodiment of the invention. Chronic wounds that may benefit from wound contraction promoted by the medicaments or methods of the invention may be selected from the group consisting of: leg ulcers; venous ulcers; diabetic ulcers; bed sores; decubitus ulcers; foot ulcers; and pressure ulcers. It will be appreciated that the long lasting nature of chronic wounds exacerbates many of the disadvantages associated with normal wound healing. For example, the duration of the period over which a patient suffering from a chronic wound will experience pain will generally be far longer than for a patient with an acute wound. Similarly the length of time over which desiccation as a result of liquid loss may occur will also be extended. Incidences of wound infection are also much increased in chronic, as opposed to acute, wounds.

Chronic wounds are also subject to many disadvantages that are not generally associated with acute wounds. For example, chronic wounds frequently expand beyond the limits of the original wounded area. This may arise as a result of infection (which may increase the damage around the margins of the wound, thereby leading to expansion) or through maceration of the tissue surrounding the wound (typically as a consequence of increased liquid loss through the chronic wound). The propensity for chronic wounds to expand beyond the boundary of the original injury means that such wounds are frequently of great surface area.

Promoting contraction of chronic wounds using the medicaments or methods of the invention may reduce the area of such wounds, and thereby facilitate their closure (either naturally, or by artificial means such as grafting or suturing). It is a preferred embodiment of the invention that the medicaments or methods of the invention be used to promote contraction of chronic wound prior to grafting or suturing of such wounds.

Pretibial lacerations are acute wounds of the leg that are very frequently slow to heal, and which frequently give rise to the development of leg ulcers. Existing treatments used for pretibial lacerations include the use of surgical procedures (such as the use of skin grafts and flaps) in an attempt to heal the wound before chronic wound development. Pretibial lacerations constitute acute wounds that may particularly benefit from treatment with the medicaments and methods of the invention, in order to promote their contraction, facilitate their closure, and thereby reduce incidences of chronic wound formation.

### Assessment of Inhibition of Scarring

The capacity to prevent, reduce or inhibit scarring has been shared between all antagonists of FXR activity investigated to date, and it is anticipated that all antagonists of FXR activity will be suitable for use in the methods and medicaments of the invention.

Preferred therapeutically effective amounts of antagonists of FXR activity (either generally, or with reference to specific selected antagonists) may be investigated using in vitro and in vivo models, and suitable assessments of efficacy made with reference to various parameters for the measurement of scarring, as described elsewhere in the specification.

In the case of inhibition of scarring that results from the healing of a wound, a suitable animal model in which the therapeutic effectiveness of an antagonist of FXR activity may be assessed, and in which a therapeutically effective amount of an antagonist of FXR activity may be determined, may involve providing the antagonist to incisional or excisional wounds of experimental animals (such as mice, rats or pigs), and assessing the scarring that results on healing of the wound.

In the case of inhibition of scarring associated with fibrotic disorders, the commonality of the biological mechanisms underlying scarring means that this scarring may also be investigated using incisional or excisional wound healing models of the type outlined above. However, the skilled person will also be aware of specific models of fibrotic disorders that may be used to further investigate the therapeutic effectiveness of antagonists of FXR in this context. For example, administration of bleomycin to experimental animals allows the generation of an experimental model of fibrosis of the lung that may be used to assess effectiveness of antagonists of FXR in the context of inhibiting scarring associated with lung fibrosis. The administration of CCl₄ to experimental animals allows the generation of an experimental model of fibrosis of the liver that may be used to assess effectiveness of antagonists of FXR in the context of inhibiting scarring associated with liver fibrosis. Furthermore, an experimental model of glomerulonephritis may be established either by injection of suitable serum proteins into an experimental animal or injection of nephrotoxic serum, and either of these animal models may be useful in assessment of antagonists of FXR activity in the inhibition of scarring associated with kidney fibrosis.

The extent of inhibition of scarring that may be required in order to achieve a therapeutic effect will be apparent to, and may readily be determined by, a clinician responsible for the care of the patient. The clinician may determine a suitable assessment of the extent of inhibition of scarring that has been achieved through utilisation of an antagonist of FXR activity in order to assess whether or not a therapeutic effect has been achieved, or is being achieved. Such an assessment may, but need not necessarily, be made with reference to suggested methods of measurement described herein.

The extent to which inhibition of scarring utilising an antagonist of FXR activity is achieved may be assessed with reference to the effects that an antagonist of FXR activity may achieve in human patients treated with the methods or medicaments of the invention. Alternatively, inhibition of scarring that may be achieved by an antagonist of FXR activity may be assessed with reference to experimental investigations using suitable in vitro or in vivo models. The use of experimental models to investigate inhibition of scarring may be particularly preferred in assessing the therapeutic effectiveness of particular antagonists of FXR activity, or in establishing therapeutically effective amounts of such an antagonist.

Animal models of scarring represent preferred experimental models for in vivo assessment of the extent of scar inhibition that may be achieved using the medicaments or methods of the invention. Suitable models may be used to specifically to investigate scarring that results from healing of a wound, and, additionally or alternatively, to investigate scarring associated with fibrotic disorders. Suitable models of both types will be known to those skilled in the art. Examples of such models are described below for illustrative purposes.

Therapeutically effective antagonists of FXR activity, and therapeutically effective amounts of such antagonists, may be selected with reference to any or all of the considerations described in the present specification.

Inhibition of scarring, using the medicaments and methods of the invention, can be effected at any body site and in any tissue or organ so far investigated. For illustrative purposes the scar inhibitory activity of medicaments and methods of the invention will primarily be described with reference to inhibition of scarring that may be brought about in the skin (the body's largest organ). However, the skilled person will immediately appreciated that many of the factors that may be relevant when considering inhibition of scarring in the skin will also be relevant to inhibition of scarring in other organs or tissues. Accordingly the skilled person will recognise that, except for where the context requires otherwise, the parameters and assessments considered below in respect of scars of the skin may also be applicable to scars of tissues other than the skin.

In the skin, treatment may improve the macroscopic and microscopic appearance of scars; macroscopically the scars may be less visible and blend with the surrounding skin, microscopically the collagen fibres within the scar may have morphology and organisation that is more similar to those in the surrounding skin. The prevention, reduction or inhibition of scarring within the context of the present invention should be understood to encompass any degree of prevention, reduction or inhibition in scarring as compared to the level of scarring occurring in a control-treated or untreated wound, or at an untreated site of a fibrotic disease (as defined above). Throughout the specification references to “prevention”, “reduction” or “inhibition” of scarring are generally to be taken, except where the context requires otherwise, to represent effectively equivalent activities, mediated by equivalent mechanisms relating to the antagonism of FXR activity, and that are all manifested in anti-scarring activity.

The inhibition of scarring achieved using methods and medicaments of the invention may be assessed and/or measured with reference to either the microscopic or macroscopic appearance of a treated scar as compared to the appearance of an untreated scar. Inhibition of scarring may also suitably be assessed with reference to both macroscopic and microscopic appearance of a treated scar.

In considering the macroscopic appearance of a scar resulting from a treated wound, the extent of scarring, and hence the magnitude of any inhibition of scarring achieved, may be assessed with reference to any of a number of parameters.

Suitable parameters for the macroscopic assessment of scars may include:
i) Colour of the scar. Scars may typically be hypopigmented or hyperpigmented with regard to the surrounding skin. Inhibition of scarring may be demonstrated when the pigmentation of a treated scar more closely approximates that of unscarred skin than does the pigmentation of an untreated scar. Similarly, scars may be redder than the surrounding skin. In this case inhibition of scarring may be demonstrated when the redness of a treated scar fades earlier, or more completely, or to resemble more closely the appearance of the surrounding skin, compared to an untreated scar.
ii) Height of the scar. Scars may typically be either raised or depressed as compared to the surrounding skin. Inhibition of scarring may be demonstrated when the height of a treated scar more closely approximates that of unscarred skin (i.e. is neither raised nor depressed) than does the height of an untreated scar.
iii) Surface texture of the scar. Scars may have surfaces that are relatively smoother than the surrounding skin (giving rise to a scar with a “shiny” appearance) or that are rougher than the surrounding skin. Inhibition of scarring may be demonstrated when the surface texture of a treated scar more closely approximates that of unscarred skin than does the surface texture of an untreated scar. Surface texture can be measured on the patient by means of profilometry, or profilometry of moulds taken from a scar.
iv) Stiffness of the scar. The abnormal composition and structure of scars means that they are normally stiffer than the undamaged skin surrounding the scar. In this case, inhibition of scarring may be demonstrated when the stiffness of a treated scar more closely approximates that of unscarred skin than does the stiffness of an untreated scar.

A treated scar will preferably exhibit inhibition of scarring as assessed with reference to at least one of the parameters for macroscopic assessment set out in the present specification. More preferably a treated scar may demonstrate inhibited scarring with reference to at least two of the parameters, even more preferably at least three of the parameters, and most preferably at least four of these parameters (for example, all four of the parameters set out above). An over-all assessment of scarring may be made using, for example, a Visual Analogue Scale or a digital assessment scale.

Suitable parameters for the microscopic assessment of scars may include:
i) Thickness of extracellular matrix (ECM) fibres. Scars typically contain thinner ECM fibres than are found in the surrounding skin. This property is even more pronounced in the case of keloid and hypertrophic scars. Inhibition of scarring may be demonstrated when the thickness of ECM fibres in a treated scar more closely approximates the thickness of ECM fibres found in unscarred skin than does the thickness of fibres found in an untreated scar.
ii) Orientation of ECM fibres. ECM fibres found in scars tend to exhibit a greater degree of alignment with one another than do those found in unscarred skin (which have a random orientation frequently referred to as “basket weave”). The ECM of pathological scars such as keloids and hypertrophic scars may exhibit even more anomalous orientations, frequently forming large “swirls” or “capsules” of ECM molecules. Accordingly, inhibition of scarring may be demonstrated when the orientation of ECM fibres in a treated scar more closely approximates the orientation of ECM fibres found in unscarred skin than does the orientation of such fibres found in an untreated scar.
iii) ECM composition of the scar. The composition of ECM molecules present in scars shows differences from that found in normal skin, with a reduction in the amount of elastin present in ECM of scars. Thus inhibition of scarring may be demonstrated when the composition of ECM fibres in the dermis of a treated scar more closely approximates the composition of such fibres found in unscarred skin than does the composition found in an untreated scar.
iv) Cellularity of the scar. Scars tend to contain relatively fewer cells than does unscarred skin. It will therefore be appreciated that inhibition of scarring may be demonstrated when the cellularity of a treated scar more closely approximates the cellularity of unscarred skin than does the cellularity of an untreated scar.

It is surprising to note that the overall appearance of scars is little influenced by the epidermal covering of the scar, even though this is the part of the scar that is seen by the observer. Instead, the inventors find that the properties of the dermis, or neo-dermis, present within the scar have greater impact on the perception of extent of scarring, as well as on the function of the scarred tissue. Accordingly assessments of criteria associated with the dermis, rather than epidermis, may prove to be the most useful in determining inhibition of scarring.

The thickness of ECM fibres and orientation of ECM fibres may be favoured parameters, for assessing inhibition of scarring. A treated scar may preferably have improved ECM orientation (i.e. orientation that is more similar to unscarred skin than is the orientation in an untreated scar).

A treated scar will preferably demonstrate inhibition of scarring as assessed with reference to at least one of the parameters for microscopic assessment set out above. More preferably a treated scar may demonstrate inhibition of scarring with reference to at least two of the parameters, even more preferably at least three of the parameters, and most preferably all four of these parameters.

The inhibition of scarring occurring at a treated wound may further be assessed with reference to suitable parameters used in the:
i) macroscopic clinical assessment of scars, particularly the assessment of scars upon a subject;
ii) assessment of photographic images of scars;
iii) assessment of silicone moulds or positive plaster casts made from silicone moulds of scars; and
iv) microscopic assessment of scars, for example by histological analysis of the microscopic structure of scars.

It will be appreciated that inhibition of scarring achieved using the medicaments or methods of the invention may be indicated by improvement of one or more of such suitable parameters, and that in the case of inhibition as assessed with reference to a number of parameters that these parameters may be combined from different assessment schemes (e.g. inhibition as assessed with reference to at least one parameter used in macroscopic assessment and at least one parameter used in microscopic assessment).

Inhibition of scarring may be demonstrated by an improvement in one or more parameters indicating that a treated scar more closely approximates unscarred skin with reference to the selected parameter(s) than does an untreated or control scar.

Suitable parameters for the clinical measurement and assessment of scars may be selected based upon a variety of measures or assessments including those described by Beausang et al (1998) and van Zuijlen et al (2002).

Typically, suitable parameters may include:
1. Assessment with Regard to Visual Analogue Scale (VAS) Scar Score.

Prevention, reduction or inhibition of scarring may be demonstrated by a reduction in the VAS score of a treated scar when compared to a control scar. A suitable VAS for use in the assessment of scars may be based upon the method described by Beausang et al. (1998). This is typically a 10 cm line in which 0 cm is considered an imperceptible scar and 10 cm a very poor hypertrophic scar.

2. Assessment with Regard to a Categorical Scale.

Prevention, reduction or inhibition of scarring may be determined by allocating scars to different categories (suitable examples of which may be “barely noticeable”, “blends well with normal skin”, “distinct from normal skin”, etc.), or by comparing a treated scar and a an untreated or control scar, noting any differences between these, and allocating the differences to selected categories (suitable examples of which may be “mild difference”, “moderate difference”, “major difference”, etc.). Assessment of this sort may be performed by the patient, by an investigator, by an independent panel, or by a clinician. Assessments of this type may be performed either directly on the patient or on photographs or moulds taken from the patient.

Inhibition of scarring may be demonstrated when an assessment indicates that treated scars are generally allocated to more favourable categories than are untreated or control scars.

### 3. Scar Height, Scar Width, Scar Perimeter, Scar Area or Scar Volume.

The height and width of scars can be measured directly upon the subject, for example by use of manual measuring devices such as callipers, or automatically with the use of profilometers. Scar width, perimeter and area may be measured either directly on the subject, by image analysis of photographs of the scar, or using plaster casts of impressions of the scar. The skilled person will also be aware of further non-invasive methods and devices that can be used to investigate suitable parameters, including silicone moulding, ultrasound, optical three-dimensional profilimetry, image analysis of photographs or photographic data from three dimensional cameras and high resolution Magnetic Resonance Imaging.

Inhibition of scarring may be demonstrated by a reduction in the height, width, area, perimeter or volume, or any combination thereof, of a treated scar as compared to an untreated scar.

4. Appearance and/or Colour of Scar Compared to Surrounding Unscarred Skin.

The appearance or colour of a treated scar may be compared to that of surrounding unscarred skin, and the differences (if any) compared with the difference between the appearance and colour of untreated scars and unscarred skin. Such a comparison may be made on the basis of a visual assessment of the respective scars and unscarred skin. The appearance of a scar may be compared with unscarred skin with reference to whether the scar is lighter or darker, or redder, than the unscarred skin. The respective colours of the scars and skin may be perfectly matched to one another, slightly mismatched, obviously mismatched or grossly mismatched.

Alternatively or additionally to visual assessment, there are a number of non-invasive calorimetric devices which are able to provide data with respect to pigmentation of scars and unscarred skin, as well as redness of the skin (which may be an indicator of the degree of vascularity present in the scar or skin). Examples of such devices include the X-rite SP-62 spectrophotometer, Minolta Chronometer CR-200/300; Labscan 600; Dr. Lange Micro Colour; Derma Spectrometer; laser-Doppler flow meter; and Spectrophotometric intracutaneous Analysis (SIA) scope.

Inhibition of scarring may be demonstrated by a smaller magnitude of difference between the appearance or colour of treated scars and unscarred skin than between untreated scars and unscarred skin.

### 5. Scar Distortion and Mechanical Performance

Scar distortion may be assessed by visual comparison of a scar and unscarred skin. A suitable comparison may categorise a selected scar as causing no distortion, mild distortion, moderate distortion or severe distortion.

The mechanical performance of scars can be assessed using a number of non-invasive methods and devices based upon suction, pressure, torsion, tension and acoustics. Suitable examples of devices capable of use in assessing mechanical performance of scars include Indentometer, Cutometer, Reviscometer, Visco-elastic skin analysis, Dermaflex, Durometer, Dermal Torque Meter and Elastometer.

Inhibition of scarring may be demonstrated by a reduction in distortion caused by treated scars as compared to that caused by untreated scars. It will also be appreciated that inhibition of scarring may be demonstrated by the mechanical performance of unscarred skin being more similar to that of treated scars than of untreated scars.

### 6. Scar Contour and Scar Texture

Scar contour may be investigated by means of visual assessment. Suitable parameters to consider in such an assessment include whether or not a scar is flush with surrounding skin, slightly proud, slightly indented, hypertrophic or keloid. The texture of a scar may be assessed with reference to the scar's appearance, and this may also be undertaken by a visual assessment as to whether the scar is, for instance, matt or shiny or has a roughened or smooth appearance as compared to unscarred skin.

Scar texture may additionally be assessed with reference to whether the scar has the same texture as unscarred skin (normal texture), is just palpable, firm or hard compared to unscarred skin. The texture of scars may also be assessed with reference to the Hamilton scale (described in Crowe et al, 1998).

In addition to the techniques set out above, there are a number of non-invasive profilimetry devices that use optical or mechanical methods for assessment of scar contour and/or texture. Such assessments may be carried out on the body of the subject or, for example, on silicone mould impressions of scars, or on positive casts made from such impressions.

Inhibition of scarring may be demonstrated in the event that treated scars have scar profiles and textures more comparable to unscarred skin than do untreated scars.

### Photographic Assessments

### Independent Lay Panel

Photographic assessment of treated and untreated scars may be performed by an independent lay panel of assessors using standardised and calibrated photographs of the scars. The scars may be assessed by an independent lay panel to provide categorical ranking data (e.g. that a given treated scar is “better”, “worse” or “no different” when compared to an untreated scar) and quantitative data using a Visual Analogue Scale (VAS) based upon the method described by Beausang et al. (1998). The capture of these data may make use of suitable software and/or electronic system(s) as described in the applicant's co-pending patent application filed as PCT/GB2005/004787.

### Expert Panel

Photographic assessment of treated and untreated scars may alternatively or additionally be performed by a panel of expert assessors using standardised and calibrated photographs of the scars to be assessed, and/or positive casts of silicone moulds. The panel of experts may preferably consist of individuals skilled in the art, suitable examples of which include plastic surgeons, dermatologists or scientists having relevant technical backgrounds.

Suitable assessment may provide categorical data, as described above, or assessments with respect to the comparison of a time-course of images of selected treated and untreated scars.

Further examples of suitable assessments that may be useful in assessing inhibition of scarring include:

Identification of the best scar within a series of scars, which may for example, be a series comprising treated scars and control and/or untreated scars. For the purposes of the present invention the best scar may be considered to be the one that most closely resembles unscarred skin. Once the best scar has been identified the magnitude of the difference between scars may be considered, for example, whether the difference between scars is mild, moderate or major. Further parameters that may be considered include the earliest time after scar formation at which a difference between scars may be detected, the time post-formation at which the difference between scars is most obvious (or alternatively the finding that the difference continues after the last timepoint assessed), as well as considering whether or not the best scar identified remains consistently better than other scars assessed.

Consideration may also be given to whether or not one scar is consistently redder than the other, and whether the redness fades over the time-points considered (or continues after the last timepoint) and if so at what time after scar formation. A suitable panel of assessors may also consider at what time after formation any difference in redness becomes detectable, as well as the time post-formation at which the difference in redness is most obvious.

A suitable panel of assessors may also consider whether or not one of a treated or untreated scar is consistently paler than the other, or paler than unscarred skin. In the event that a difference in paleness is detectable consideration may be given to the time after scar formation at which the difference may be detected, the time at which the difference is most obvious, and the time at which the difference disappears.

A further parameter that may be assessed by a suitable panel of assessors is the texture of treated and untreated scars. In comparing treated and untreated scars the panel may consider which of the scars has the texture most similar to that of unscarred skin, the earliest time after scar formation at which any difference in texture may be detected, the time post formation at which any difference is most obvious, and the time at which any difference disappears

Comparison of treated and untreated scars may further assess which of the scars is narrowest, and which of the scars is shortest. Consideration may also be given to the shape of the scar and the proportion of the scar margin that is distinguishable from the surrounding skin. As with previously described visual assessments and assessments of colour, the presence, degree and location of hyper-pigmentation may also be considered.

### Clinical Assessment

A clinician, or an independent panel of clinicians may assess the scar(s) on a patient using any of the forgoing parameters e.g. VAS, colour, categorical scales, etc. A suitable clinician may be a clinician responsible for care of a patient, or may be a clinician investigating efficacy of therapies for inhibition of scarring.

### Patient Assessment

A patient may assess their own scars and/or compare scars by means of a structured questionnaire. A suitable questionnaire may measure parameters such as: the patient's satisfaction with their scar; how well the scar blends with the unscarred skin; as well as the effect of the scar on their daily life (suitable questions may consider whether the patient uses clothes to hide the scar, or otherwise avoids exposing it); scar symptoms (examples of which may include itch, pain or paresthesia). Inhibition of scarring may be indicated by the treated scar receiving a more positive rating from the patient, and/or causing the patient fewer problems, and/or causing fewer or less scar symptoms, and/or an increase in patient satisfaction compared to an untreated scar.

### Microscopic Analysis

Microscopic assessment may also provide a suitable means by which the quality of treated and untreated or control scars may be compared. Microscopic assessment of scar quality may typically be carried out using histological sections of scars. The process of microscopically assessing and measuring scars may take into consideration categorical data based on suitable parameters, which may include the following:

1. Collagen organisation. In assessing collagen organisation reference may be made to the orientation of collagen fibres present in the scar, the density of such fibres and collagen fibre thickness in the papillary and reticular dermis. An inhibition of scarring may be indicated when a treated scar contains collagen organisation that more closely approximates that found in unwounded skin than does the organisation in untreated or control treated scars.
2. Abundance of ECM components. Scars typically contain an increased amount of ECM components such as collagen when compared to unscarred skin. An inhibition of scarring may be indicated when a treated scar contains reduced abundance of ECM components when compared to untreated or control treated scars, or when a treated scar contains an abundance of ECM components that is more similar to unscarred skin than the abundance contained in an untreated or control scar.
3. Visual analogue scale (VAS) assessment of collagen organisation and abundance in the papillary dermis and the reticular dermis may also provide a useful index of scar quality. Inhibition of scarring may be indicated when the quality of a treated scar is closer to that of unscarred skin than is the quality of an untreated or control scar.
4. Angiogenesis and Inflammation. Consideration may be given to the number of blood vessels present, the size of the blood vessels present and evidence of inflammation, including an assessment of any level of inflammation present. An inhibition of scarring may be indicated when a treated scar contains blood vessels and inflammatory cells in quantities and arrangements that more closely approximate those found in unwounded skin than those found in untreated or control treated scars.

Other features that may be taken into account in assessing the microscopic quality of scars include elevation or depression of the scar relative to the surrounding unscarred skin, and the prominence or visibility of the scar at the interface with the unscarred skin.

It will be appreciated that the assessments described above allow the generation of scar ranking data which is able to provide an indication as to whether a treated scar is better, worse or no different compared to a control, untreated or other suitable comparator scar.

In addition to categorical data, quantitative data (preferably relating to the above parameters) can be generated using image analysis in combination with suitable visualisation techniques. Examples of suitable visualisation techniques that may be employed in assessing scar quality are specific histological stains or immuno-labelling, wherein the degree of staining or labelling present may be quantitatively determined by image analysis

Quantitative data may be usefully and readily produced in relation to the following parameters:
1. Scar width, height, elevation, volume and area.
2. Collagen organisation, collagen fibre thickness, collagen fibre density.
2. Number and orientation of fibroblasts.
4. Quantity and orientation of other ECM molecules e.g. elastin, fibronectin.
5. The number and distribution of inflammatory cells present.
6. The number and distribution of blood vessels present.

Prevention, reduction or inhibition of scarring may be demonstrated by a change in any of the parameters considered above such that a treated scar more closely resembles unscarred skin than does a control or untreated scar (or other suitable comparator).

Preferably inhibition of scarring may be demonstrated with reference to more than one parameter. More preferably inhibition of scarring may be demonstrable with reference to both a clinical (i.e. observed on the subject) parameter and a photographic parameter. Even more preferably inhibition of scarring may be demonstrable with reference to a clinical parameter, a photographic parameter, and also a microscopic assessment parameter (for instance a histological parameter). Most preferably inhibition of scarring may be demonstrable with reference to a clinical VAS score, external lay panel VAS score and ranking (from photographic images) and microscopic VAS score of the reticular dennis.

The assessments and parameters discussed above are suitable for assessment of the effects of antagonists of FXR activity as compared to control, placebo or standard care treatment in animals or humans. It will be appreciated that these assessments and parameters may be utilised in determining therapeutically effective antagonists of FXR activity, and in determining therapeutically effective amounts of such antagonists. Appropriate statistical tests may be used to analyse data sets generated from different treatments in order to investigate significance of results.

Many of the parameters described above for the assessment of scarring have previously been viewed as primarily suitable for the assessment of scarring that results from healing of a wound. However, the inventors believe that many of these parameters are also suitable for assessment of scarring associated with fibrotic disorders. Additional or alternative parameters that may be considered when assessing scarring associated with fibrotic disorders will be apparent to the skilled person. The following examples are provided by way of illustration only.

Scarring associated with fibrotic disorders may be assessed with reference to trichrome staining (for example Masson's trichrome or Mallory's trichrome) of biopsy samples taken from a tissue believed to be subject to the fibrotic disorder. These samples may be compared with non-scarred tissues that have been taken from tissues not subject to the fibrotic disorder, and with reference tissues representative of staining in the same tissue (or a range of tissues) subject to different extents of scarring associated with the fibrotic disorder. Comparisons of such tissues may allow assessment of the presence and extent of scarring associated with a fibrotic disorder that is present in the tissue of interest. Protocols for trichrome staining are well known to the skilled person, and kits that may be used to conduct trichrome staining are commercially available.

It will be appreciated that in many cases it may be preferred to avoid invasive procedures such as the collection of biopsies. In recognition of this fact a number of non-invasive procedures have been devised that allow assessment of scarring associated with fibrotic disorders without the need for biopsy samples. Examples of such procedures include Fibrotest (FT) and Actitest (AT).

These commercially available assays use five or six biochemical markers of scarring associated with fibrotic disorders for use as a non-invasive alternative to liver biopsy in patients with chronic hepatitis C or B, alcoholic liver disease and metabolic steatosis (for instance the overweight, patients with diabetes or hyperlipidemia). Through use of such biochemical markers, and analysis using selected algorithms, these procedures are able to determine levels of liver fibrosis and necroinflammatory activity. The use of such tests is increasingly clinically accepted as an alternative to biopsies, and the tests are commercially available from suppliers such as BioPredictive.

It will be appreciated by the skilled person that the methods described above may be used to allow assessment of scarring that is associated with one or more fibrotic disorders in order to determine whether or not prevention, reduction or inhibition of such scarring utilising the medicaments or methods of the invention would be advantageous. Furthermore, scar assessment methods of the type described above may be used to determine therapeutically effective antagonists of FXR activity suitable for inhibition of scarring associated with a fibrotic disorder, as well as determining therapeutically effective amounts of such antagonists.

### Assessment of Wound Contraction

Promotion of wound contraction may be most readily demonstrated by an increase in the rate at which wound area decreases. The increase in the rate of wound contraction that may be achieved using the medicaments or methods of the invention should be distinguished from the rate at which the wound is covered with a new epithelial layer (in the skin a new epidermis), which is related to the rate of re-epithelialisation.

An increase in the rate at which wound area decreases will indicate that the healing of the wound in question has been accelerated. The rate at which the area of a treated wound decreases may be compared with control wounds, or with reference data regarding the rate at which area of untreated or control wounds decreases in order to assess any difference in the rates observed.

The area of a wound may be readily assessed macroscopically. Suitable assessment may be performed directly on the wound itself (e.g. direct measurement of the area of a wound), or on measurements taken from captured images or photographs of a wound. In assessing wound area it is important that the area measured is that between the margins of the wound, rather than measurement of the area of the portion of wound that has not been covered by re-epithelialisation. Given the potential for confusion in this matter it may frequently be preferred that wound area in experimental circumstances is assessed microscopically through use of histological slides (in which the position of the margins of the wound may be clearly distinguished from the position of the leading edge of re-epithelialisation).

When wound area is to be assessed microscopically, this may most readily be achieved by determining the width of the wound. Promotion of wound contraction may be demonstrated by an increase in the rate at which the width of a treated wound decreases as compared to the rate at which width of a control or untreated wound decreases.

The rate at which wound width decreases thus provides a preferred indication of the rate at which contraction of the wound is occurring. The skilled person will appreciate that increased wound contraction, leading to the formation of narrower wounds, is important in the acceleration of healing, and provides the advantage that wound size is rapidly decreased. This decrease in wound width may be particularly beneficial in the context of wounds that are to be healed by primary intention, since the margins of the relatively narrow wounds may be readily apposed (and then held in apposition by sutures, or the like). Accordingly it will be appreciated that the use of the medicaments or methods of the invention in connection with wounds that are to be healed by primary intention represents a preferred embodiment of the invention.

In the case of microscopic assessment of wound width, this may be undertaken using suitable histological slides. Preferably wound width may be measured at a standardised “reference” point within the wound. The inventors have found that measurements taken midway through the depth of the wound allow for an accurate and reproducible assessment of wound width. Suitable image analysis software may aid the assessment of wound width in this manner.

Macroscopic assessment of wound width may either be performed directly (i.e. with measurements taken directly from a wound), or indirectly, in which case measurements may be taken using representations of the wound, such as photographs, traced outlines, mouldings, or the like. Image analysis software may be useful in the macroscopic assessment of wound width, particularly as assessed from photographs.

The inventors believe that a treated wound in which wound contraction has been promoted may have a healing age that is greater than the chronological age of the wound (the actual time elapsed since formation of the wound).

The use of antagonists of FXR activity to promote wound contraction in accordance with the present disclosure may preferably lead to a treated wound having a “healing age” that is at least a day faster than an untreated wound, preferably at least five days faster, more preferably at least ten days faster; yet more preferably at least eleven, twelve, thirteen, fourteen of fifteen days faster, still more preferably fifteen or more days faster, and most preferably 20 (or more) days faster.

### Preferred Routes of Administration and Suitable Formulations

Preferred routes of administration, by which therapeutically effective amounts of an antagonist of FXR activity may be administered to a tissue in which it is desired to inhibit scarring and/or to promote wound contraction, are discussed more fully elsewhere in the specification, but it may generally be preferred that therapeutically effective antagonists are provided by local administration to the tissue where they are to have their effect. Suitable methods by which such local administration may be achieved will depend on the identity of the tissue in question, and may also be influenced by whether the tissue where they are to have their activity is a scar or wound. Preferred routes of administration may include local injection (for example intradermal injection in the case where it is wished to inhibit scarring and/or promote wound contraction of the skin). Other suitable means of administration include the use of topical medicaments such as sprays; powders; drops; ointments or creams; or release from local devices e.g. stents, implants, polymers, biomaterials, or skin replacement materials. Examples of solid or liquid medicaments of the invention, and suitable formulations that may be used are considered elsewhere in the specification.

Generally, medicaments of the invention may be formulated and manufactured in any form that allows for the medicament to be administered to a patient such that a therapeutically effective amount of an antagonist of FXR activity is provided to a site where scarring is to be inhibited and/or wound contraction promoted.

Scarring associated with fibrotic disorders will frequently occur in relatively inaccessible tissues and organs, and these organs may also constitute sites where it may be wished to promote wound contraction. In these cases (inhibition of scarring associated with a fibrotic disorder, or promotion of wound contraction in inaccessible tissues) the antagonist of FXR activity be administered systemically. Suitable routes of administration include, without limitation, oral, transdermal, inhalation, parenteral, sublingual, rectal, vaginal and intranasal administration. By way of example, solid oral formulations (such as tablets or capsules) providing a therapeutically effective amount of an antagonist of FXR activity may be used for the inhibition of scarring associated with renal fibrosis or cirrhosis of the liver. Aerosol formulations for inhalation may be preferred as a means for providing antagonists of FXR activity in the event that it is wished to inhibit scarring associated with chronic obstructive pulmonary disease or other fibrotic disorders of the lungs and airways.

It will be appreciated that many of the routes of administration described above may also be suitable for topical administration to a tissue in which it is wished to inhibit scarring and/or promote wound contraction (for example, inhalation or intranasal administration for promoting wound contraction and/or inhibiting scarring in the respiratory system).

Medicaments of the invention may preferably be provided in the form of one of more dosage units providing a therapeutically effective amount (or a known fraction or multiple of a therapeutically effective amount) of an antagonist of FXR activity. Methods of preparing such dosage units will be well known to the skilled person; for example see Remington's Pharmaceutical Sciences 18^{th} Ed. (1990).

The methods or medicaments of the invention may be used prophylactically, i.e. prior to wound or scar formation. For example, methods or medicaments of the invention may be utilised prior to wounding or prior to the onset of a fibrotic disorder.

In the case of prophylactic promotion of wound contraction and/or inhibition of scarring associated with healing of a wound, this may involve administration of a therapeutically effective amount of an antagonist of FXR activity at sites where no wound presently exists, but where a wound is to be formed. By way of example, a therapeutically effective amount of an antagonist of FXR activity may be administered to sites that are to undergo wounding as a result of elective procedures (such as surgery), or to sites that are believed to be at elevated risk of wounding.

It may be preferred that the medicaments of the invention are administered to the site around the time of wounding, or immediately prior to the forming of a wound (for example in the period up to six hours before wounding) or the medicaments may be administered at an earlier time before wounding (for example up to 48 hours before a wound is formed). The skilled person will appreciate that the most preferred times of administration prior to formation of a wound will be determined with reference to a number of factors, including the formulation and route of administration of the selected medicament, the dosage of the medicament to be administered, the size and nature of the wound to be formed, and the biological status of the patient (which may be determined with reference to factors such as the patient's age, health, and predisposition to healing complications or adverse scarring), as well as the half-life of the selected antagonist. The prophylactic use of methods and medicaments in accordance with the invention is a preferred embodiment of the invention, and is particularly preferred in the prevention, reduction or inhibition of scarring in the context of surgical wounds.

In the case of the inhibition of scarring associated with fibrotic disorders, medicaments of the invention may be administered to a site at elevated risk of developing a fibrotic disorder prior to formation of said disorder. Suitable sites may be those that are perceived to be at elevated risk of the development of fibrotic disorders. An elevated risk of development of fibrotic disorders may arise as a result of disease, or as a result of environmental factors (including exposure to fibrotic agents), or as a result of genetic predisposition.

When used for the inhibition of scarring associated with a fibrotic disorder, a therapeutically effective amount of an antagonist of FXR activity may be administered immediately prior to onset of a fibrotic disorder, or at an earlier time. The skilled person will be able to establish the optimal time for administration of medicaments of the invention used to treat fibrotic disorders using standard techniques well known to those skilled in the art, and familiar with the clinical progression of scarring associated with fibrotic disorders.

The methods and medicaments of the invention are also able to promote wound contraction and/or inhibit scarring if administered after a wound has already been formed. It is preferred that such administration should occur as early as possible after formation of the wound, but agents of the invention are able to inhibit scarring and/or promote wound contraction at any time up until the healing process has been completed (i.e. even in the event that a wound has already partially healed the methods and medicaments of the invention may be used to promote contraction and/or inhibit scarring in respect of the remaining un-healed portion). It will be appreciated that the “window” in which the methods and medicaments of the invention may be used to inhibit scarring and/or promote contraction is dependent on the nature of the wound in question (including the degree of damage that has occurred, and the size of the wounded area). Thus, in the case of a large wound, the methods and medicaments of the invention may be administered relatively late in the healing response yet still be able to inhibit scarring and/or promote contraction, as a consequence of the relatively prolonged time that large wounds require to heal.

The methods and medicaments of the invention may, for instance, preferably be administered within the first 24 hours after a wound is formed, but may still inhibit scarring and/or promote contraction if administered up to ten, or more, days after wounding.

Similarly, the methods and medicaments of the invention may be administered to a site at which a fibrotic disorder is already developing, in order to prevent further scarring associated with the fibrotic disorder taking place. This use will obviously be advantageous in situations in which the degree of scarring that has occurred prior to administration of the antagonist of FXR activity is sufficiently low that the fibrotic tissue is still able to function.

Medicaments of the invention may preferably be administered within 24 hours of the onset of scarring associated with a fibrotic disorder, but may still be effective if administered considerably later in the fibrotic process. For example, medicaments may be administered within a month of the onset of the fibrotic disorder (or of the diagnosis that scarring associated with the fibrotic disorder is taking place), or within sixth months, or even one or more years, depending on the extent of scarring that has already occurred, the proportion of the tissue effected by the fibrotic disorder, and the rate at which the fibrotic disorder is progressing.

The methods and medicaments of the invention may be administered on one or more occasions (as necessary) in order to inhibit scarring and/or promote wound contraction.

For instance, in the case of inhibition of scarring that results from the healing of a wound, therapeutically effective amounts of an antagonist of FXR activity may be administered to a wound as often as required until the healing process has been completed. A therapeutically effective amount of an antagonist of FXR activity may be administered to a wound as often as required until wound contraction has been promoted to a desired extent. By way of example, the medicaments of the invention may be administered daily or twice daily to a wound for at least the first three days following the formation of the wound. The inventors have found that regimes involving two administrations of medicaments of the invention, the first prior to formation of a wound and the second after wounding, are particularly beneficial in inhibiting scar formation and/or promoting wound contraction. Preferably such regimes may involve a first administration immediately prior to formation of a wound and a second administration 24 hours after wounding.

Most preferably the methods or medicaments of the invention may be administered both before and after formation of a wound. The inventors have found that administration of the medicaments of the invention immediately prior to the formation of a wound, followed by daily administration of an antagonist of FXR activity for one or more days following wounding, is particularly effective in promoting wound contraction, and/or inhibiting scarring resulting from the healing of a wound or associated with a fibrotic disorder.

In the case where an antagonist of FXR activity is to be used to inhibit scarring associated with a fibrotic disorder, a therapeutically effective amount of the antagonist of FXR activity may be provided by means of a number of administrations. Suitable regimes may involve administration monthly, weekly, daily or twice daily.

The inventors believe that therapeutically effective amounts of an antagonist of FXR activity may also be used to reduce existing scars. This is applicable to existing scars that result from the healing of a wound, and/or existing scars associated with fibrotic disorders. Accordingly the use of methods and medicaments of the invention in the reduction of existing scars constitutes a preferred use according to the invention. A therapeutically effective amount of an antagonist of FXR activity may be provided by means of any number of suitable administrations. Suitable regimes for these administrations may be readily devised by the skilled person using techniques (including in vitro studies, animal and human studies) well known in and established within the pharmaceutical industry.

For the purposes of the present specification by “agent” or “agent of the invention” is meant a therapeutically effective antagonist of FXR activity. It will be appreciated that all such suitable agents may be incorporated in medicaments in accordance with the invention, and all may be used in the methods or uses of the invention. The medicaments of the invention represent preferred compositions by which a therapeutically effective amount of an antagonist of FXR activity may be administered in order to put the methods of the invention into practice.

It will be appreciated that the amount of a medicament of the invention that should be provided to a wound, scar or fibrotic disorder (or to a site where a wound or fibrotic disorder will occur), in order that a therapeutically effective amount of an antagonist of FXR activity may be administered, depends on a number of factors. These include the biological activity and bioavailability of the agent present in the medicament, which in turn depends, among other factors, on the nature of the agent and the mode of administration of the medicament. Other factors in determining a suitable therapeutic amount of a medicament may include:
A) The half-life of the agent in the subject being treated.
B) The specific condition to be treated (e.g. acute wounding or chronic fibrotic disorders).
C) The age of the subject.
D) The size of the site to be treated.

The frequency of administration will also be influenced by the above-mentioned factors and particularly the half-life of the chosen agent within the subject being treated.

Generally when medicaments in accordance with the invention are used to treat existing wounds or scars (whether resulting from healing of a wound, or associated with a fibrotic disorder) the medicament should be administered as soon as the wound occurs or as early as possible in the development of the scar (for instance, as soon as the fibrotic disorder begins). In the case of wounds or fibrotic disorders that are not immediately apparent, such as those at internal body sites, medicaments may be administered as soon as the wound or disorder, and hence the risk of scarring, is diagnosed. Therapy with methods or medicaments in accordance with the invention should continue until scarring has been inhibited, and/or wound contraction promoted, to a clinician's satisfaction.

Frequency of administration will depend upon the biological half-life of the agent used. Typically a cream or ointment containing an agent of the invention should be administered to a target tissue such that the concentration of the agent at a wound or site of fibrosis is maintained at a level suitable to inhibit scarring and/or promote wound contraction. This may require administration daily or even several times daily. The inventors have found that administration of an agent of the invention immediately prior to wounding, with a further administration one day after wounding is particularly effective to promote wound contraction and/or inhibit scarring that would otherwise result from the healing of such a wound.

Medicaments of the invention, may be administered by any suitable route capable of achieving the desired effect of inhibiting scarring and/or promoting wound contraction but it is preferred that the medicaments be administered locally at a wound site or site of a fibrotic disorder.

The inventors have found that scarring may be inhibited and/or wound contraction promoted by the administration of an agent of the invention by injection at a wound site or site of a fibrotic disorder. For instance, in the case of skin wounds or skin fibrosis, agents of the invention may be administered by means of intradermal injection. Thus a preferred medicament in accordance with the invention comprises an injectable solution of an agent of the invention (e.g. for injection around the margins of a wound, or at a site likely to be wounded). Suitable formulations for use in this embodiment of the invention are considered below.

Alternatively, or additionally, medicaments of the invention may also be administered in a topical form to promote wound contraction and/or inhibit scarring (whether resulting from the healing of a wound, or associated with a fibrotic disorder). In the case of promoting wound contraction and/or inhibiting scarring that would otherwise result from healing of a wound, such administration may be effected as part of the initial and/or follow up care for the wounded area.

The inventors have found scarring can be inhibited and/or wound contraction promoted by topical application of an agent of the invention to a wound or fibrotic disorder (or, in the case of prophylactic application, to a tissue or site where a wound or fibrotic disorder will occur).

Compositions or medicaments containing agents of the invention may take a number of different forms depending, in particular, on the manner in which they are to be used. Thus, for example, they may be in the form of a liquid, ointment, cream, gel, hydrogel, powder or aerosol. All of such compositions are suitable for topical application to a site of requiring antagonism of FXR activity (for example, either a wound, scar or a fibrotic disorder), and this represents a preferred means of administering agents of the invention to a subject (person or animal) in need of treatment.

The agents of the invention may be provided on a sterile dressing or patch, which may be used to cover a wound or fibrotic site where scarring is to be inhibited and/or wound contraction promoted.

The agents of the invention may be released from a device or implant, or may be used to coat such a device e.g. a stent or controlled release device e.g. wound dressing.

It will be appreciated that the vehicle of a composition comprising agents of the invention should be one that is well tolerated by the patient and allows release of the agent to the wound, scar or fibrotic site. Such a vehicle is preferably biodegradeable, bioresolveable, bioresorbable and/or non-inflammatory.

Medicaments and compositions comprising agents of the invention may be used in a number of ways. Thus, for example, a composition may be applied in and/or around a wound or fibrotic disorder in order to promote wound contraction and/or inhibit scarring. If the composition is to be applied to an existing wound or fibrotic site, then the pharmaceutically acceptable vehicle will be one which is relatively “mild” i.e. a vehicle which is biocompatible, biodegradable, bioresolvable and non-inflammatory.

An agent of the invention, or a nucleic acid encoding such an agent (as considered further below), may be incorporated within a slow or delayed release device. Such devices may, for example, be placed on or inserted under the skin and the agent or nucleic acid may be released over days, weeks or even months.

Delayed release devices may be particularly useful for patients, such as those suffering from extensive or pathological scarring or from long-lasting scarring associated with a fibrotic disorder or from chronic wounds requiring contraction, who require long-term administration of therapeutically effective amounts of an antagonist of FXR activity. Such devices may be particularly advantageous when used for the administration of an agent or nucleic acid that would otherwise normally require frequent administration (e.g. at least daily administration by other routes).

Daily doses of an agent of the invention may be given as a single administration (e.g. a daily application of a topical formulation or a daily injection). Alternatively, the agent of the invention may require administration twice or more times during a day. In a further alternative, a slow release device may be used to provide optimal doses of an agent of the invention to a patient without the need to administer repeated doses.

A dose of a composition comprising agents of the invention may preferably be sufficient to provide a therapeutically effective amount of an antagonist of FXR activity in a single administration. However, it will be appreciated that each dose need not in itself provide a therapeutically effective amount of an antagonist of FXR activity, but that a therapeutically effective amount may instead be built up through repeated administration of suitable doses.

Various suitable forms are known for compositions comprising agents of the invention. In one embodiment a pharmaceutical vehicle for administration of an agent of the invention may be a liquid and a suitable pharmaceutical composition would be in the form of a solution. In another embodiment, the pharmaceutically acceptable vehicle is a solid and a suitable composition is in the form of a powder or tablet. In a further embodiment the agent of the invention may be formulated as a part of a pharmaceutically acceptable transdermal patch.

A solid vehicle can include one or more substances that may also act as flavouring agents, lubricants, solubilizers, suspending agents, fillers, glidants, compression aids, binders or tablet-disintegrating agents; it can also comprise an encapsulating material. In powders, the vehicle is a finely divided solid that is in admixture with the finely divided agent of the invention. In tablets, the agent of the invention is mixed with a vehicle having the necessary compression properties in suitable proportions and compacted in the shape and size desired. The powders and tablets preferably contain up to 99% of the agent of the invention. Suitable solid vehicles include, for example, calcium phosphate, magnesium stearate, talc, sugars, lactose, dextrin, starch, gelatin, cellulose, polyvinylpyrrolidine, low melting waxes and ion exchange resins.

Liquid vehicles may be used in preparing solutions, suspensions, emulsions, syrups, elixirs and pressurized compositions. The agent of the invention can be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilizers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). A solution comprising 1% v/v ethanol in PBS is a particularly preferred vehicle for use in compositions comprising the antagonist of FXR activity guggulsterone (Z). For parenteral administration, the vehicle can be an oily ester such as ethyl oleate and isopropyl myristate. Sterile liquid vehicles are useful in sterile liquid form compositions for parenteral administration. The liquid vehicle for pressurized compositions can be halogenated hydrocarbon or other pharmaceutically acceptable propellant.

Liquid pharmaceutical compositions which are sterile solutions or suspensions can be utilized by, for example, intramuscular, intrathecal, epidural, intraperitoneal, intradermal, intrastromal (cornea), intraadventitial (blood vessels) or subcutaneous injection. Sterile solutions can also be administered intravenously. The agent of the invention may be prepared as a sterile solid composition that may be dissolved or suspended at the time of administration using sterile water, saline, or other appropriate sterile injectable medium (such as PBS). Vehicles are intended to include necessary and inert binders, suspending agents, lubricants and preservatives.

In the situation in which it is desired to administer an agent of the invention by means of oral ingestion, it will be appreciated that the chosen agent will preferably be an agent having an elevated degree of resistance to degradation. For example, the agent of the invention may be protected (using the techniques well known to those skilled in the art) so that its rate of degradation in the digestive tract is reduced.

As set out elsewhere in the specification, compositions of agents of the invention are suitable for use in inhibiting scarring in the eye (and particularly in the cornea or retina), and in some circumstances to promote wound contraction in the eye. Scarring of the cornea may result from corneal wounds, which may be caused by trauma to the cornea arising as a result of accidental injury or as a result of surgical operations (e.g. laser surgery on the cornea). In the case of administration of agents of the invention to the outer surfaces of the eye, such as the cornea, a preferred medicament of the invention may be in the form of an eye drop.

Scarring in the eye may also be associated with fibrotic disorders such as proliferative vitreoretinopathy. In the event that it is wished to inhibit scarring associated with fibrotic disorders such as proliferative vitreoretinopathy, it may be preferred to administer a therapeutically effective amount of an antagonist of FXR activity by means of intravitreal injection or localised (e.g. intraocular) release device. Such injections may preferably follow surgery or intravitreal implantation procedures.

Agents of the invention may be used to inhibit scarring, or to promote contraction, in a range of “internal” wounds or fibrotic disorders (i.e. wounds or fibrotic disorders occurring within the body, rather than on an external surface). Examples of internal wounds include penetrative wounds that pass through the skin into underlying tissues, and wounds associated with surgical procedures conducted within the body. The range of fibrotic disorders that effect internal sites is extensive, and includes lung fibrosis, liver fibrosis, kidney fibrosis and muscle fibrosis.

In a preferred example, medicaments in accordance with the invention for use in the inhibition of scarring, or promotion of wound contraction, in the lungs or other respiratory tissues may be formulated for inhalation.

In a preferred example, medicaments in accordance with the invention for use in the inhibition of scarring, or promotion of wound contraction, in the body cavities e.g. abdomen, pelvis may be formulated as a lavage, gel or instillate.

Known procedures, such as those conventionally employed by the pharmaceutical industry (e.g. in vivo experimentation, clinical trials etc), may be used to establish specific formulations of compositions comprising agents of the invention and precise therapeutic regimes for administration of such compositions (such as daily doses of the active agent and the frequency of administration) that may be used to inhibit scarring or promote wound contraction.

A suitable dose of an agent in accordance with the invention able to inhibit scarring, or promote wound contraction, may depend upon a range of factors including (but not limited to) the nature of the tissue to be treated, the area and/or depth of the wound or fibrosis to be treated, the severity of the wound or fibrosis, and the presence or absence of factors predisposing to pathological scar or chronic wound formation.

The inventors believe that the amount of an antagonist of FXR activity that may be administered to a wound or site of fibrosis in a single incidence of treatment may preferably be in the region 0.3 pmoles to 3.2 μmoles/cm of wound or cm² of wound or fibrosis, more preferably in the region of 3.2 pmoles to 32 pmoles/cm of wound or cm² of wound or fibrosis.

For the purposes of the present disclosure, a linear centimetre of wound may be taken to comprise a site where a linear centimetre of wound is to be formed, as well as a wounded site.

A centimetre of wound in the context of the present disclosure constitutes a unit by which the size of a wound to be treated may be measured. A centimetre of wound may be taken to comprise any square centimetre of a body surface that is wounded in whole or in part. For example, a wound of two centimetres length and one centimetre width (i.e. with a total surface area of two centimetres²) will be considered to constitute “two wound centimetres”, while a wound having a length of two centimetres and a width of two centimetres (i.e. a total surface area of four centimetres²) will constitute four wound centimetres. By the same token, a linear wound of two centimetres length, but of negligible width (i.e. with negligible surface area), will, for the purposes of the present invention, be considered to constitute “two wound centimetres”, if it passes through two square centimetres of the body surface.

The size of a wound in wound centimetres should generally be assessed when the wound is in its relaxed state (i.e. when the body site bearing the wounded area is in the position adopted when the body is at rest). In the case of skin wounds, the size of the wound should be assessed when the skin is not subject to external tension.

By way of further example, the preferred amount of an antagonist of FXR activity to be administered to a wound or site of fibrosis over a period of approximately 24 hours may be in the region of approximately 3 pmoles to 32 pmoles/cm of wound or cm² fibrosis (if administered by injection), or 3 pmoles to 3.2 μmoles/cm of wound or cm² fibrosis (if administered topically).

The skilled person will appreciate that the suggestions above are provided for guidance. In particular it will be appreciated that the amount of an antagonist of FXR activity to be administered via topical administration may be altered depending on permeability of the tissue or organ to which the topical composition is administered. Thus in the case of relatively impermeable tissues or organs it may be preferred to increase the amount of the antagonist of FXR activity to be administered. Such an increased amount of an antagonist of FXR activity may still represent a therapeutically effective amount, if the amount of the agent taken up into the tissue or organ where scarring is to be inhibited, or wound contraction promoted, is therapeutically effective (i.e. if a therapeutically effective amount permeates the tissue or organ where scarring is to be inhibited, irrespective of the fact that a larger, non-therapeutic, amount of the agent may remain on the surface of, and unable to penetrate, the tissue or organ being treated).

The inventors believe that the amount of an antagonist of FXR activity (such as guggulsterone (Z)) to be administered to a wound or site of fibrosis in a single incidence of treatment will preferably not exceed 3.2 nmoles/cm of wound, or cm² of wound or fibrosis. More preferably the amount administered in a single incidence of treatment will be less than 321 pmoles/cm of wound, or cm² of wound or fibrosis, and most preferably it will be less than 64 pmoles/cm of wound, or cm² of wound or fibrosis.

Preferably, the amount of an antagonist of FXR activity (such as guggulsterone (Z)) to be administered to a wound or site of fibrosis over a period of approximately 24 hours will not exceed 3.2 nmoles/cm of wound, or cm² of wound or fibrosis. More preferably the amount to be administered over a period of approximately 24 hours will be less than 321 pmoles/linear cm of wound, or cm of wound or fibrosis, and most preferably it will be less than 64 pmoles/linear cm of wound, or cm² of wound or fibrosis.

Preferably the total amount of an antagonist of FXR activity (such as guggulsterone (Z)) administered to a wound or site of fibrosis will not exceed 3.2 nmoles/cm of wound, or cm² of wound or fibrosis. More preferably the total amount will not exceed 321 pmoles/cm of wound, or cm² of wound or fibrosis, and most preferably it will not exceed 64 pmoles/cm of wound, or cm² of wound or fibrosis.

The total amount of an antagonist of FXR activity (such as guggulsterone (Z)) that may be administered by local injection to a wound or site of fibrosis may be in the region of 0.32 pmoles to 3.2 nmoles/centimetre of wound or cm² of wound or fibrosis.

In the case of topical application of an antagonist of FXR activity (such as guggulsterone (Z)) to wounds or sites of fibrosis, a suitable amount to be administered may be in the region of 0.32 pmoles to 0.32 μmoles/cm of wound or cm² of wound or fibrosis.

An antagonist of FXR activity (such as guggulsterone (Z)) may preferably be provided in the form of an injectable solution at a concentration of between 3.2 nM and 32 μM. Preferably approximately 100 μL of such a solution administered per centimetre of wound or cm² of wound or fibrosis over a 24 hour period.

An antagonist of FXR activity (such as guggulsterone (Z)) may more preferably be administered as a 32 nM to 3.2 μM solution, or even more preferably as a solution of approximately 32 nM to approximately 320 nM. 100 μL of such a solution may be administered per centimetre of wound or cm² of wound or fibrosis over a 24 hour period.

Most preferably the antagonist of FXR activity (such as guggulsterone (Z)) may be administered as a 321 nM solution with 100 μL of such a solution administered per centimetre of wound or cm² of wound or fibrosis over a 24 hour period.

The skilled person will appreciate that effective therapeutic amounts of an antagonist of FXR activity may be determined with reference to the concentration of the agent that is attained in the organ or tissue to which they are administered. The information regarding therapeutically effective dosages set out herein will provide sufficient guidance to allow the skilled person to calculate the local concentrations of an antagonist of FXR activity established by intradermal injection, and, based on these values, to determine suitable amounts of such agents that may be administered by other routes in order to achieve equivalent local concentrations.

The inventors have found that guggulsterone (Z) may be administered by way of an injectable solution containing between 1 ng/100 μL and 1000 ng/100 μL in order to inhibit scarring, promote wound contraction, or treat fibrosis when administered as an intradermal injection providing 100 μL of solution per linear cm of wound margin or cm² of wound or fibrosis.

It will be appreciated that the guidance as to doses and amounts of an antagonist of FXR activity to be used provided above is applicable both to medicaments of the invention, and also to the methods of the invention.

In the case where the paragraphs above consider the administration of a specified amount of a medicament per linear cm of a wound it will be appreciated that this volume may be administered to either one or both of the margins of a wound to be treated (i.e. in the case of a reference to 100 μl of a medicament, this may be administered as 100 μl to the wound margins, or as 50 μl to each of the wound margins to be joined together).

The skilled person will recognise that the information provided in the preceding paragraphs as to amounts of an antagonist of FXR activity which may be administered to wounds or sites of fibrotic disorders in order to inhibit scarring, and/or promote wound contraction, may be varied by the skilled practitioner in response to the specific clinical requirements of an individual patient. For example, it will be appreciated that in the case of particularly deep or wide wounds the amounts provided by way of guidance above may be varied upwards, while still providing a therapeutically effective amount of an antagonist of FXR activity. Suitable variations based on the guidance provided above will be readily apparent to those of skill in the art.

Medicaments of the invention may be used to inhibit scarring and/or promote wound contraction as a monotherapy (e.g. through use of medicaments of the invention alone). Alternatively the methods or medicaments of the invention may be used in combination with other compounds or treatments for the inhibition of scarring. Suitable compounds that may be used as parts of such combination therapies will be well known to those skilled in the art.

The skilled person will appreciate that therapeutically effective amounts of an antagonist of FXR activity may be administered at the sites of wounds or fibrotic disorders where it is wished to inhibit scarring or treat fibrosis by virtue of cellular expression (commonly referred to as gene therapy). Suitable means by which such antagonists of FXR activity may be expressed at therapeutically effective levels may be readily determined by those skilled in the art. It will be appreciated that gene silencing techniques represent particularly suitable techniques for use in such embodiments, and that cells engineered to express antagonists of FXR activity, or nucleic acid constructs encoding such antagonists are suitable for use in the medicaments and methods of the invention.

Accordingly, the invention provides a method of inhibiting scar formation, the method comprising inducing cellular expression of a therapeutically effective amount of an antagonist of FXR activity at a site where scarring is to be inhibited. It will be a matter of routine experimentation for one skilled in the art to devise protocols by which cells may be induced to express therapeutically effective amounts of an antagonist of FXR activity.

The invention will now be further described by way of example with reference to the following experimental protocols and studies, and the accompanying Figures in which:

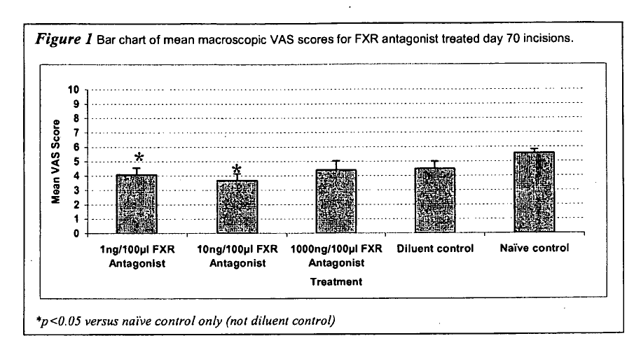

FIG. 1 compares macroscopic VAS scores of treated scars, diluent control treated scars and naïve control scars. Results are shown in the form of a bar chart showing macroscopic VAS scores for scars formed 70 days after wounding. “*” indicates p<0.05 versus naïve control.

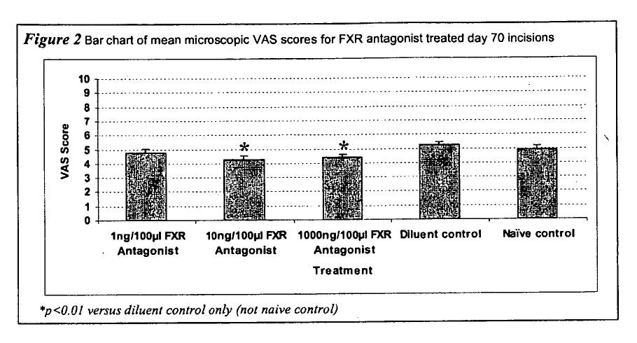

FIG. 2 compares microscopic VAS scores of treated scars, diluent control treated scars and naïve control scars. Results are shown in the form of a bar chart showing microscopic VAS scores for scars formed 70 days after wounding. “*” indicates p<0.05 versus diluent control.

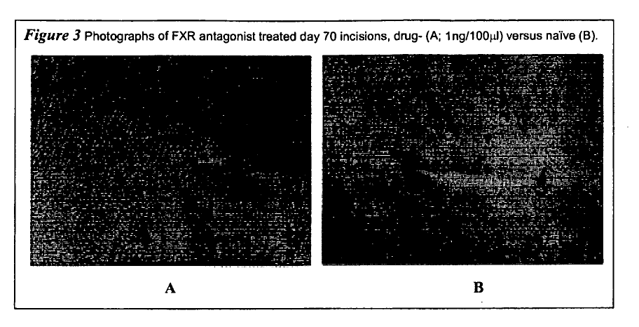

FIG. 3 shows representative photographs comparing the macroscopic appearances of treated scars and naïve control scars. The images were taken 70 days after wounding.

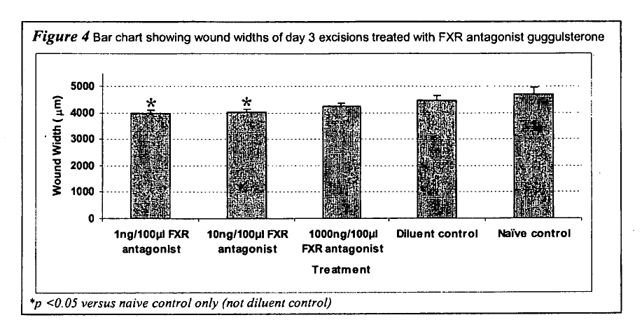

FIG. 4 compares widths of treated excisional wounds, diluent control treated excisional wounds, and naïve control excisional wounds. The results are shown in the form of a bar chart showing measurements of wound width taken three days after wounding. “*” indicates p<0.05 versus naïve control.

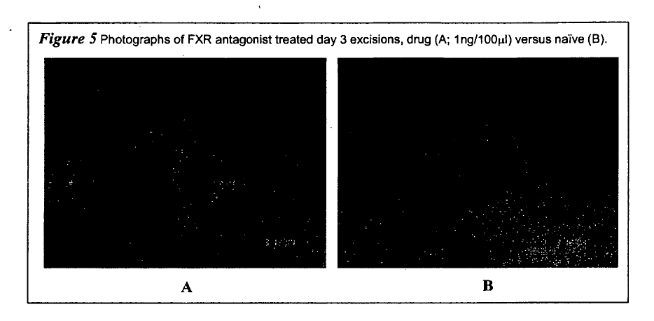

FIG. 5 shows representative photographs comparing the macroscopic appearances of treated excisional wounds and naïve control excisional wounds. The images were taken three days after wounding.

### EXPERIMENTAL RESULTS

The inventors investigated the effects of antagonists of FXR activity on scarring and wound contraction using in an in vivo model of scarring and wound healing.

Incisional Scarring and Wound Healing Model and Treatment with an Antagonist of FXR Activity

Guggulsterone (Z) (Catalogue number C370690), an antagonist of FXR activity, was purchased from Calbiochem.

The guggulsterone (Z) was diluted in a solution containing 1% v/v ethanol (absolute ethanol, 200 proof, for molecular biology, purchased from Sigma-Aldrich) in phosphate buffered saline (PBS) to produce three solutions having concentrations as follows:
1. 1 ng/100 μl (a 32 nM solution);
2. 10 ng/100 μl (a 321 nM solution); and
3. 1000 ng/100 μl (a 32 μM solution).

1% v/v ethanol/PBS (EtOH/PBS), but without guggulsterone (Z), was used as a diluent control.

### Scarring and Wounding Model, Dosing & Harvest Timepoint

### Day 3

At day 0, Male Sprague Dawley rats (200-250 g) were anaesthetised, shaved and wound sites were marked according to the Renovo rat incisional and excisional wounding template (4 wound model, 2×1 cm wounds at 5 cm from the base of the skull and 1 cm from the midline in each rat and 2×5 mm excisions at 8.5 cm from the base of the skull and 1 cm from the midline of each rat). One hundred microlitres of Guggulsterone (Z) at 1 ng, 10 ng or 1000 ng in 1% v/v ethanol/PBS (EtOH/PBS), were injected intradermally at the target wound sites, A, B, C and D. The intradermal injections caused the formation of a raised bleb, which was then either immediately incised to form 1 cm long experimental wounds at wound positions A and B, or subject to a 5 mm punch biopsy, at wound positions C and D. A separate group of rats were wounded, without any injection, to act as the untreated Naïve control group in addition to the diluent injected control group. All treated wounds were re-injected again 1 day post-wounding with the appropriate treatment via either injection of 501 to each of the two margins of the 1 cm wound, or via injection of 25 μl to each of the four quadrants of a 5 mm punch biopsy, and harvested at day 3 post-wounding. The wounds were photographed after wounding, prior to re-injection on day 1 and on day of harvest. The wounds were assessed using standard macroscopic wound assessment sheets that include a Visual Analogue Scale (VAS) to quantify the quality of the wounds. The excisions and incisions were analysed microscopically, after histological processing by image analysis for re-epithelialisation (excisions only) and wound width (incisions only) to determine the role of Guggulsterone (Z) on early wound healing.

At Day 0, male Sprague Dawley rats (200-250 g) were anaesthetised, shaved and wound sites were marked according to the Renovo rat incisional wounding template (2 wound model, 2×1 cm wounds at 5 cm from the base of the skull and 1 cm from the midline in each rat). One hundred microlitres of Guggulsterone (Z) at 1 ng, 10 ng or 1000 ng in 1% v/v ethanol/PBS (EtOH/PBS), were injected intradermally at the target wound sites, A and B. The intradermal injections caused the formation of a raised bleb, which was then immediately incised to form 1 cm long experimental wounds at wound positions A and B. A separate group of rats were wounded, without any injection, to act as the untreated Naïve control group in addition to the diluent injected control group. All treated wounds were re-injected again 1 day post-wounding with the appropriate treatment via injection of 50 μl to each of the two margins of the 1 cm wound and harvested at day 70 post-wounding. The wounds were photographed after wounding, prior to re-injection on day 1 and on day of harvest. The scars were assessed using standard macroscopic scar assessment sheets that include a Visual Analogue Scale (VAS) to quantify the quality of the scars. Accordingly, each injection of the 1 ng/100 μl solution provided 3.2 pmoles of guggulsterone (Z), whilst each injection of the 10 ng/100 μl solution provided 32 pmoles of guggulsterone (Z), and each injection of the 1000 ng/100 μl solution provided 3.2 nmoles of guggulsterone (Z).

Diluent control treated wounds received PBS/EtOH (administered in the same volume and by the same route as guggulsterone (Z) solutions in treated wounds), and the naïve control wounds received no treatment.

### Assessment of Scarring

70 days after wounding the experimental rats were killed, and the scars resulting from treated wounds and control wounds assessed both macroscopically and microscopically.

The scars of the experimental rats were photographed and assessed using macroscopic scar assessment sheets. Macroscopic assessment of scarring was carried out using a visual analogue scale (VAS) consisting of a 0-10 cm line representing a scale, from left to right, of 0 (corresponding to normal skin) to 10 (indicative of a bad scar). A mark was made by an assessor on the 10 cm line based on an overall assessment of the scar taking into account parameters such as the height, width, contour and colour of the scar. The best scars (typically of small width with colour, height and contour like normal skin) were scored towards the normal skin end of the scale (the left hand side of the VAS line) and bad scars (typically large width, raised with uneven contours and whiter colour) were scored towards the bad scar end of the scale (the right hand side of the VAS line). The marks were measured from the left hand side to provide the final value for the scar assessment in centimetres (to 1 decimal place).

For microscopic assessment the scars were excised from the experimental rats (incorporating a small amount of surrounding normal tissue) and fixed in 10% (v/v) buffered formal saline. The fixed tissue was then processed for wax histology. Histological slides were stained using Masson's trichrome, and scarring assessed, by two independent observers, using a microscopic visual analogue scale (VAS). This consisted of a 0-10 cm line representing a scale, from left to right, of 0 (corresponding to normal skin) to 10 (indicative of a bad scar). A mark was made on the 10 cm line based on an overall assessment of the scar taking into account parameters such as collagen fibre spacing, orientation and thickness. The best scars (typically narrow scars with thick and randomly organised collagen fibres that have normal spacing between fibres, similar to the surrounding normal dermis) were scored towards the normal skin end of the scale (the left hand side of the VAS line) and bad scars (typically wide scars with thin densely packed parallel collagen fibres) were scored towards the bad scar end of the scale (the right hand side of the VAS line). The marks were measured from the left hand side to provide the final value for the scar assessment in centimetres (to 1 decimal place).

A comparison of the macroscopic VAS scores of scars resulting from healing of guggulsterone (Z) treated wounds, diluent control treated wounds and naïve control wounds is shown in FIG. 1.

A comparison of the microscopic VAS scores of scars formed on healing of guggulsterone (Z) treated wounds, diluent control treated wounds and naïve control wounds is shown in FIG. 2.

Representative images showing the macroscopic appearance of scars formed on healing of guggulsterone (Z) treated wounds and naïve control wounds are shown in FIG. 3.

### Assessment of Acceleration of Wound Healing

All histological assessments of early wound healing events were made using preserved 5 micron-thickness wound sections taken from the widest part of each excised biopsy site. Sections were stained with Haematoxylin and Eosin to aid visualisation of structural features, and measurements made using image analysis software. The total wound width was taken as a linear measurement from the top of the excisional biopsy between the two wound margins. The average wound width value was calculated from all of the wounds at each treatment group.

A comparison of the widths of guggulsterone (Z) treated excisional wounds, diluent control treated excisional wounds and naïve control excisional wounds, all measured at three days after wounding, is shown in FIG. 4.

Representative images showing the appearance of guggulsterone (Z) treated excisional wounds and naïve control excisional wounds three days after wounding are shown in FIG. 5.

### Results

### Inhibition of Scarring

Both macroscopic and microscopic analysis of scars formed from incisional wounds (assessed at 70 days post-wounding) showed that the addition of guggulsterone (Z), an antagonist of FXR activity, to wounds was able to significantly inhibit scarring. This ability to inhibit scarring was observed in respect of each concentration of guggulsterone (Z) investigated.

Administration of the antagonist of FXR activity guggulsterone (Z) at a concentration of either 1 ng/100 μl or 10 ng/100 μl (respectively equivalent to a total dose of 3.2 pmoles or 32 pmoles of guggulsterone (Z) per linear cm of wound) significantly inhibited scarring, as compared to controls (p<0.05), when assessed using a macroscopic VAS. This significant improvement in scar appearance thus clearly illustrates the suitability of antagonists of FXR activity for use in the therapeutic inhibition of scarring.

That scarring is effectively inhibited by use of a therapeutically effective amount of an antagonist of FXR activity is clearly illustrated in FIG. 3, which shows representative macroscopic images of a treated scar and naïve control scar. The scar resulting from a wound treated with a therapeutically effective amount an antagonist of FXR activity (guggulsterone (Z)) is considerably more difficult to detect than the scar produced on healing of a naïve control wound.

The results show that administration of an antagonist of FXR activity (such as guggulsterone (Z)) is capable of inhibiting scarring. These results also provide guidance as to how preferred therapeutically effective amounts of such an antagonist may be determined.

Given the similarities between the biological mechanisms involved in scarring that results from healing of a wound and scarring associated with fibrotic disorders the results reported above provide a clear indication that therapeutically effective amounts of an antagonist of FXR activity may be utilised in the prevention, reduction or inhibition of both scarring resulting from wounds and scarring associated with fibrotic disorders.

### Promotion of Wound Contraction

Administration of the antagonist of FXR activity guggulsterone (Z) was able to promote wound contraction, as assessed by measurement of width of excisional wounds three days after wound formation.

In particular, administration of the antagonist of FXR activity guggulsterone (Z) at a concentration of either 1 ng/100 μl or 10 ng/100 μl (respectively equivalent to a total dose of 3.2 pmoles or 32 pmoles of guggulsterone (Z) per cm of wound) significantly decreased wound width, as compared to controls (p<0.05), when excisional wounds were assessed at three days after wounding. The ability of antagonists of FXR activity to decrease wound width in this manner demonstrates their ability to promote wound contraction. Further, the results provided indicate that antagonists of FXR activity may be used to promote wound contraction (and thereby accelerate wound healing) while also inhibiting scarring.

1. The use of an antagonist of FXR activity in the manufacture of a medicament for the prevention, reduction or inhibition of scarring.

2. The use of an antagonist of FXR activity in the manufacture of a medicament for the promotion of wound contraction.

3. The use according to claim 1 or claim 2, wherein the antagonist of FXR activity is selected from the group consisting of guggulsterone (Z); guggulsterone (E); a scalarane; 80-574; and a 5α-bile alcohol.

4. The use according to claim 1 or claim 3, wherein the scarring is scarring that results from healing of a wound.

5. The use according to claim 2 or claim 4, wherein the scarring occurs in a tissue selected from the group consisting of: the skin; the eye; blood vessels; tendons, ligaments or muscle; the oral cavity, lips and palate; the liver; the heart; digestive tissues; reproductive tissues; the central nervous system; the peripheral nervous system; the abdominal cavity; the pelvic cavity and the thoracic cavity.

6. The use according to claim 1 or claim 3, wherein the scarring is associated with a fibrotic disorder.

7. The use according to claim 6, wherein the fibrotic disorder is selected from the group consisting of: skin fibrosis; scleroderma; progressive systemic fibrosis; lung fibrosis; muscle fibrosis; kidney fibrosis; glomerulosclerosis; glomerulonephritis; uterine fibrosis; renal fibrosis; cirrhosis of the liver, liver fibrosis; adhesions, such as those occurring in the abdomen, pelvis or spine; chronic obstructive pulmonary disease; fibrosis following myocardial infarction; central nervous system fibrosis following a stroke, fibrosis associated with neurodegenerative disorders; fibrosis associated with proliferative vitreoretinopathy (PVR); restenosis; endometriosis; ischemic disease and radiation fibrosis.

8. The use according to any preceding claim, wherein the medicament is for use in the prevention, reduction or inhibition of scarring, and/or promotion of wound contraction, in the skin.

9. The use according to any one of claims 1 to 7, wherein the medicament is for use in the prevention, reduction or inhibition of scarring in the eye.

10. The use according to any one of claims 1 to 7, wherein the medicament is for use in the prevention, reduction or inhibition of adhesions, such as those occurring in the abdomen, pelvis or spine.

11. The use according to any preceding claim, wherein the medicament is a topical medicament.

12. The use according to any preceding claim, wherein the medicament is an injectable solution.

13. The use according to claim 12, wherein the medicament is for intradermal injection.

14. The use according to any preceding claim, wherein the medicament provides up to 32 μM of the antagonist of FXR activity per linear cm of wound, or cm² of a wound or fibrotic disorder, over a 24 hour period.

15. A method of preventing, reducing or inhibiting scarring, the method comprising administering a therapeutically effective amount of an antagonist of FXR activity, to a patient in need of such prevention, reduction or inhibition.

16. A method of promoting wound contraction, the method comprising administering a therapeutically effective amount of an antagonist of FXR activity, to a patient in need of such promoted wound contraction.

17. A method according to claim 15 or claim 16, wherein the therapeutically effective amount of the antagonist of FXR activity is administered by means of a medicament manufactured in accordance with any of claims 1 to 14.

18. A method according to claim 15, 16 or 17, wherein the scarring is scarring that results from the healing of a wound.

19. A method according to any one of claims 15 to 18, wherein the scarring is associated with a fibrotic disorder.
